(19) United States
(12) Patent Application Publication
(10) Pub. No.: US 2003/0211991 A1
(43) Pub. Date: Nov. 13, 2003

(54) Human sez6 nucleic acids and polypeptides

(76) Inventor: Eric Wen Su, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/257,904

(22) PCT Filed: Apr. 17, 2001
(86) PCT No.: PCT/US01/10809

(51) Int. Cl. C07K 14/705; C07H 21/04; C12P 21/02; C12N 5/06

(52) U.S. Cl. 514/12; 435/69.1; 435/320.1; 435/325; 536/23.5; 530/350; 800/8

(57) ABSTRACT

The present invention relates to human SEZ6 (hSEZ6) polypeptides, and isolated nucleic acids that encode at least one hSEZ6 polypeptide. Vectors, host cells, transgenics, and chimeric mammals comprising hSEZ6 polynucleotides and/or polypeptides, as well as methods of making and using thereof, and hSEZ6-specific antibodies are included in the present invention.

### BACKGROUND OF THE INVENTION

1. Field of the Invention

The present invention relates to compounds and compositions comprising novel human SEZ6 (hSEZ6) polypeptides, nucleic acids, host cells, transgenics, chimerics, antibodies, compositions, and methods of making and using thereof.

2. Related Art

The navigation of axons to their targets is a critical step in the patterning of neuronal projections. The growth cones at the tips of developing axons are thought to select appropriate pathways by recognizing distinct guidance markers present in their environment. There is substantial evidence that axonal guidance depends on expression of both attractive and repulsive molecules on cells and in the extracellular matrix along the pathway of advancing axons (Dodd et al., Neuron, 1:105-116 (1988); Harrelson and Goodman, Science, 242:700-708 (1988); Tessier-Lavigne and Goodman, Science, 247:1123-1133 (1996)).

The semaphorins are the largest family of repulsive axon guidance molecules. Secreted and trans-membrane semaphorins are widely expressed in neuronal and non-neuronal tissues throughout development and into adulthood. Recently, the differentially expressed axonal glycoproteins neuropilin-1 and neuropilin-2 have been shown to be cell surface receptors for the secreted semaphorins Sema III and Sema E/IV, respectively (He and Tessier-Lavigne, Cell, 90:739-751 (1997);); Kolodkin, A. L., et al., Cell, 90:753-762 (1997); Chen, H., et al., Neuron, 19:547-559 (1997)). Although multiple splice variants of neuropilin-2 exist that result in proteins having distinct structural arrangements, the neuropilins, in general, are characterized as having large extracellular domains, a transmembrane domain, and a short cytoplasmic domain. The extracellular domains of neuropilins are complex, usually consisting of two so-called CUB (extracellular complement-binding) domains, two domains having homology to coagulation factors V and VIII (b domains), and a so-called MAM (meprin, A5, μ) domain. All of these domains have been implicated in mediating protein-protein interactions. More specifically, MAM domains are known to mediate homophilic interactions in receptor tyrosine phosphatases (Bork, P., and Beckmann, G., J. Mol. Biol., 231:539-545 (1993); Zondag, G. C., et al., J. Biol. Chem., 270:14247-14250 (1995)). Recent studies have shown that neuropilins can assemble both homo-and heterophilically into dimers or multimers and that this assembly is mediated through MAM domain interactions (Chen, H., et al., Neuron, 21:1283-1290 (1998)). Furthermore, it has been reported that the specificity of ligand binding is defined by the pocket created by the CUB and b domains (Chen, H., et al., (1998)). Accordingly, antibodies raised against the extra-cellular domain of neuropilin-1 and/or neuropilin-2 block the repulsive and collapse-inducing activities of Sema III and Sema E/IV, respectively, on sensory axons (He and Tessier-Lavigne, (1997); Kolodkin, (1997)).

SEZ6 was originally cloned from a mouse cerebral cortex cDNA library and its expression was shown to be brain specific and up-regulated in mice treated with pentylentetrazole (PTZ), one of the convulsant drugs. (Shimizu-Nishikawa, Keiko, et al., Mol. Brain Res., 28:201-210 (1995).

Structurally, SEZ6 typically appears to be a membrane protein with numerous potential N-linked glycosylations sites, five copies of short consensus repeat (SCR) and two repeated sequences which are partially similar to CUB domains. (Shimizu-Nishikawa, K., et al., Biochem. Biophys. Res. Com., 216(1):382-389 (1995)). SCR is widely known as a characteristic structure of the super-family of complement C3b/C4b binding proteins even though many non-complement proteins also have SCRs. Most proteins that have SCRs are also involved in protein-protein interaction. The presence of both SCRs, in addition to the CUB motifs, in SEZ6 polypeptides supports its putative role as a neuronal adhesion molecule structurally similar to the neuropilins. Like the neuropilins, SEZ6 splice variants which encode various secreted and membrane bound isoforms of SEZ6 polypeptides have also been identified. (Shimizu-Nishikawa, K., et al., (1995)).

In view of the importance of neuronal adhesion molecules in neural development and dysfunction there is a need to provide human SEZ6 (hSEZ6) polypeptides, nucleic acids, and host cells, transgenics, chimerics, comprising human SEZ6 nucleic acids and polypeptides. Accordingly, we provide a human homolog of the mouse SEZ6 cDNA as well as methods of making and using hSEZ6 nucleic acids and polypeptides.

### SUMMARY OF THE INVENTION

The present invention provides isolated hSEZ6 nucleic acids and encoded hSEZ6 polypeptides, including fragments and/or variants thereof, as well as hSEZ6 compositions, probes, primers, vectors, host cells, antibodies, transgenics, chimerics and methods of making and using thereof, as described and enabled herein.

The present invention provides, in one aspect, isolated nucleic acid molecules comprising a polynucleotide, or a complementary polynucleotide, encoding hSEZ6 polypeptides, as well as fragments or variants, comprising at least one domain thereof.

The present invention further provides recombinant vectors, comprising 1-40 of said isolated hSEZ6 nucleic acid molecules of the present invention, host cells containing said nucleic acids and/or said recombinant vectors.

The present invention also provides methods of making or using hSEZ6 nucleic acids, and/or vectors, host cells, and transgenic animals comprising said nucleic acids.

The present invention also provides an isolated hSEZ6 polypeptide, comprising at least one fragment, domain, or specified variant having at least 90-100% identity to the contiguous amino acids of at least one portion of at least one of SEQ ID NOS:3-11. Examples of functional fragments of preference include polypeptides comprising SEQ ID NO:3 wherein said polypeptide lacks from 1 to 50 amino acid residues from the amino terminus of SEQ ID NO:3 or from 1 to 260 amino acid residues from the carboxy-terminus of SEQ ID NO:3. More preferable functional fragments are polypeptides comprising SEQ ID NO:3 wherein said polypeptide lacks from 10 to 25 amino acid residues from the amino-terminus of SEQ ID NO:3 and from 1 to 260 amino acid residues from the carboxy-terminus of SEQ ID NO:3. A most preferred functional fragment is a polypeptide as shown in SEQ ID NO:4.

In another embodiment the present invention relates to an isolated protein molecule, or functional fragment thereof, wherein said protein molecule comprises the sequence identified as SEQ ID NO:3.

The present invention also provides an isolated hSEZ6 polypeptide as described herein, wherein the polypeptide further comprises at least one specified substitution, insertion, or deletion of one or more portion or one or more specific residues corresponding to at least one polypeptide sequence as shown in SEQ ID NOS:3-11.

The present invention also provides an isolated hSEZ6 polypeptide as described herein, wherein the polypeptide has at least one activity such as, but not limited to, promoting or inhibiting neurite outgrowth and/or neurite adhesion (Kolodkin, A., et al., Neuron, 21:1079-1092, (1998); Kolodkin, et al., (1997); Wilson et al., J. Cell Sci. 109:3129-3138 (1996); Pimenta et al., Neuron, 15:287-297 (1995)), inducing neural regeneration, inhibiting neural degeneration, preventing seizures, reducing frequency and/or severity of seizures, promoting or inhibiting primary or secondary sexual development, and altering behavioral patterns including, but not limited to, sleep and eating disorders. An hSEZ6 polypeptide can therefore be screened for such activities according to known methods. An hSEZ6 polypeptide can thus be screened for a corresponding activity according to these and other methods known in the art.

The present invention also provides an isolated nucleic acid probe, primer, or fragment, as described herein, wherein the nucleic acid comprises a polynucleotide of at least 10 nucleotides, corresponding or complementary to at least 10 nucleotides of at least one of SEQ ID NOS:1 or 2.

The present invention also provides a recombinant vector comprising an isolated hSEZ6 nucleic acid as described herein.

The present invention also provides a host cell, comprising an isolated hSEZ6 nucleic acid as described herein.

The present invention also provides a method for constructing a recombinant host cell that expresses an hSEZ6 polypeptide, comprising introducing into the host cell an hSEZ6 nucleic acid in replicatable form as described herein to provide the recombinant host cell. The present invention also provides a recombinant host cell provided by a method as described herein.

The present invention also provides a method for expressing at least one hSEZ6 polypeptide in a recombinant host cell, comprising culturing a recombinant host cell as described herein under conditions wherein at least one hSEZ6 polypeptide is expressed in detectable or recoverable amounts.

The present invention also provides an isolated hSEZ6 polypeptide produced by a recombinant, synthetic, and/or any purification method as described herein and/or as known in the art.

The present invention also provides an hSEZ6 antibody, or fragment thereof, comprising a polyclonal and/or monoclonal antibody, or fragment thereof, that specifically binds at least one epitope specific to at least one hSEZ6 polypeptide as described herein.

The present invention also provides a method for producing an hSEZ6 antibody or antibody fragment, comprising generating the antibody or fragment thereof that binds at least one epitope that is specific to an isolated hSEZ6 polypeptide as described herein, the generating done by known recombinant, synthetic and/or hybridoma methods.

The present invention also provides an hSEZ6 antibody or fragment thereof produced by a method as described herein or as known in the art.

The present invention also provides a composition comprising an isolated hSEZ6 nucleic acid and/or poly-peptide as described herein and a carrier or diluent. The carrier or diluent can optionally be pharmaceutically acceptable, according to known methods.

Methods for treatment of diseases or disorders using the nucleic acids, polypeptides, antibodies, vectors, host cells, and/or transgenic cells described herein are also part of the invention. For instance, a method of treatment or prophylaxis for a nervous disease or disorder can be effected with the polypeptides, nucleic acids, antibodies, vectors, host cells, transgenic cells, and/or compositions described. Similarly, included in the present invention are methods for the prophylaxis or treatment of pathophysio-logical conditions of the nervous system in which at least one cell type involved in said condition is sensitive or responsive to a polypeptide, nucleic acid, antibody, host cell, transgenic cell, or composition of the present invention. The present invention includes methods for treatment when the condition to be treated involves peripheral nervous system nerve damage, central nervous system nerve damage; neurodegenerative disorders; abnormal primary or secondary sexual development; undesired reproductive disorders including, but not limited to, impotence, infertility, or reduced libido; and undesired behavioral disorders including, but not limited to, sleep or eating disorders. In any of these cases, prophylaxis or treatment comprises administering an effective amount of the polypeptide, nucleic acid, antibody, host cell, transgenic cell, or pharmaceutically acceptable formulation thereof, to a vertebrate. Preferably, the vertebrate is a mammal. Most preferably, the vertebrate is a human.

The present invention also provides a method for identifying compounds that bind an hSEZ6 polypeptide, comprising

a) admixing at least one isolated hSEZ6 polypeptide as described herein with a test compound or composition; and

b) detecting at least one binding interaction between the polypeptide and the compound or composition, optionally further comprising detecting a change in biological activity, such as a reduction or increase.

The present invention also provides methods for identifying polypeptides that bind an hSEZ6 polypeptide which comprises use of at least one isolated hSEZ6 polypeptide as described herein in at least one protein-protein interaction assays or reporter systems known in the art.

### DESCRIPTION OF THE INVENTION

Citations

All publications or patents cited herein are entirely incorporated herein by reference as they show the state of the art at the time of the present invention to provide description and enablement of the present invention. Publications refer to scientific, patent publication or any other information available in any media format, including all recorded, electronic or printed formats. The following citations are entirely incorporated by reference: Ausubel, et al., eds., Current Protocols in Molecular Biology, John Wiley & Sons, N.Y., N.Y. (1987-1998); Coligan et al., eds., Current Protocols in Protein Science, John Wiley & Sons, Inc., N.Y., N.Y. (1995-1999); Sambrook, et al., Molecular Cloning: A Laboratory Manual, 3^{rd} Edition, Cold Spring Harbor, N.Y. (2001); Harlow and Lane, Antibodies, a Laboratory Manual, Cold Spring Harbor, N.Y. (1989); Coligan, et al., eds., Current Protocols in Immunology, John Wiley & Sons, N.Y., N.Y. (1992-1999); Gennaro, Ed., Remington's Pharmaceutical Sciences, 18^{th} Edition, Mack Publishing Co. (Easton, Pa.) 1990.

Definitions

The following definitions of terms are intended to correspond to those as well known in the art. The following terms are therefore not limited to the definitions given, but are used according to the state of the art, as demonstrated by cited and/or contemporary publications or patents.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, etc.; and chemically synthesized compounds having properties known in the art to be characteristic of amino acids.

The incorporation of non-natural amino acids, including synthetic non-native amino acids, substituted amino acids, or one or more D-amino acids into the hSEZ6 polypeptides of the present invention (“D-hSEZ6 polypeptides”) is advantageous in a number of different ways. D-amino acid-containing polypeptides may exhibit increased stability in vitro or in vivo compared to L-amino acid-containing counterparts. Thus, the construction of polypeptides incorporating D-amino acids can be particularly useful when greater stability is desired or required in vivo. More specifically, D-peptides may be more resistant to endogenous peptidases and proteases, thereby providing improved bioavailability of the molecule, and prolonged lifetimes in vivo when such properties are desirable. When it is desirable to allow the peptide to remain active for only a short period of time, the use of L-amino acids therein will permit endogenous peptidases, proteases to digest the molecule, thereby limiting the cell's exposure to the molecule. Additionally, D-peptides cannot be processed efficienty for major histocompatibility complex class II-restricted presentation to T helper cells, and are therefore less likely to induce humoral immune responses in the whole organism.

In addition to using D-amino acids, those of ordinary skill in the art are aware that modifications in the amino acid sequence of a peptide, polypeptide, or protein can result in equivalent, or possibly improved, second generation peptides, etc., that display equivalent or superior functional characteristics when compared to the original amino acid sequences. Alterations in the hSEZ6 polypeptides of the present invention can include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) as the hSEZ6 analog sequences disclosed herein.

One factor that can be considered in making such changes is the hydropathic index of amino acids. The importance of the hydropathic amino acid index in conferring interactive biological function on a protein has been discussed by Kyte and Doolittle [J. Mol. Biol. 157: 105-32 (1982)]. It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in the hSEZ6 polypeptides of the present invention can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally-occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

It should be noted that changes that are not expected to be advantageous can also be useful if these result in the production of functional sequences. Since small peptides, can be easily produced by conventional solid phase synthetic techniques, the present invention includes novel utility for peptides, such as those discussed herein, containing the amino acid modifications discussed above, alone or in various combinations. To the extent that such modifications can be made while substantially retaining the activity of the peptide, their utility is included within the scope of the present invention. The utility of such modified peptides, can be determined without undue experimentation by, for example, the methods described herein.

While biologically functional equivalents of the present hSEZ6 polypeptides can have any number of conservative or non-conservative amino acid changes that do not significantly affect their activity(ies), or that increase or decrease activity as desired, 40, 30, 20, 10, 5, or 3 changes, or any range or value therein, may be preferred. In particular, 10 or fewer amino acid changes may be preferred; more preferably, seven or fewer amino acid changes may be preferred; most preferably, five or fewer amino acid changes may be preferred. The encoding nucleotide sequences (gene, plasmid DNA, cDNA, synthetic DNA, or mRNA, for example) will, thus, have corresponding base substitutions, permitting them to code on expression for the biologically functional equivalent forms of the hSEZ6 polypeptides. In any case, preferred hSEZ6 peptides, polypeptides, or proteins exhibit the same or similar biological or immunological activity(ies) as that(those) of the hSEZ6 polypeptides specifically disclosed herein, or increased or reduced activity, if desired. The activity(ies) of ant variant hSEZ6 polypeptides can be determined by the methods described herein. Variant hSEZ6 polypeptides are biologically functionally equivalent to those specifically disclosed herein and may have activity(ies) differing from those of the presently disclosed molecules by about ±50% or less, preferably by about ±40% or less, more preferably by about ±30% or less, more preferably by about ±20% or less, and even more preferably by about ±10% or less, when assayed by the methods disclosed herein.

The terms “complementary” or “complementarity” as used herein refer to the capacity of purine, pyrimidine, synthetic or modified nucleotides to associate by partial or complete complementarity through hydrogen or other bonding to form partial or complete double- or triple-stranded nucleic acid molecules. The following base pairs occur by complete complementarity: (i) guanine (G) and cytosine (C); (ii) adenine (A) and thymine (T); and adenine (A) and uracil (U). “Partial complementarity” refers to association of two or more bases by one or more hydrogen bonds or attraction that is less than the complete complementarity as described above. Partial or complete complementarity can occur between any two nucleotides, including naturally occurring or modified bases, e.g., as listed in 37 CFR §1.822. All such nucleotides are included in polynucleotides of the invention as described herein.

A “therapeutically-effective amount” is the minimal amount of active agent (e.g., an hSEZ6 polypeptide) which is necessary to impart therapeutic benefit to a mammal. For example, a “therapeutically-effective amount” to a mammal suffering or prone to suffer from a medical disorder is such an amount which induces, ameliorates or otherwise causes an improvement in the pathological symptoms, disease progression, physiological conditions associated with or resistance to succumbing to the aforementioned disorders.

An “effective amount” is the minimal amount of active agent (e.g., an hSEZ6 polypeptide) which is necessary to invoke a detectable biological consequence.

The term “fusion protein” denotes a hybrid protein molecule not found in nature comprising a translational fusion or enzymatic fusion in which two or more different proteins or fragments thereof are covalently linked on a single polypeptide chain. The term “polypeptide” also includes such fusion proteins.

“Human SEZ6” or “hSEZ6” refers to a nucleic acid, gene, cDNA (e.g. SEQ ID NO:1), fragments thereof, and/or to any polypeptide sequence (e.g., SEQ ID NO:2) encoded thereby. The term “hSEZ6” without further limitation refers to both the native hSEZ682 polypeptide (SEQ ID NO:3) as well as the mature form of the hSEZ682 polypeptide which is predicted to be as shown in SEQ ID NO:3. If not stated otherwise, the term “hSEZ682 polypeptide” encompasses the full-length and any fragments of the hSEZ6 polypeptide as shown in SEQ ID NO:2, as well as, secreted, mature, fused, variant, alternatively spliced, and allelic forms thereof.

“Host cell” refers to any eucaryotic, procaryotic, or fusion or other cell or pseudo cell or membrane-containing construct that is suitable for propagating and/or expressing an isolated nucleic acid that is introduced into a host cell by any suitable means known in the art (e.g., but not limited to, transformation or transfection, or the like), or induced to express an endogenous nucleic acid encoding an hSEZ6 polypeptide according to the present invention. The cell can be part of a tissue or organism, isolated in culture or in any other suitable form.

The term “human antibody” includes antibodies having variable and constant regions corresponding to human germline immunoglobulin sequences as described by Kabat et al. (1991). Human antibodies are generated by various methods now routine to one skilled in the art. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. Any human antibody can also be substituted at one or more positions with an amino acid, e.g., a biological property enhancing amino acid residue, which is not encoded by the human germline immunoglobulin sequence. In preferred embodiments, these replacements are within the CDR regions as described in detail below.

Human antibodies have at least three potential advantages over non-human and chimeric antibodies for use in human therapy:

1) because the effector portion of the antibody is human, it may interact better with the other parts of the human immune system (e.g., destroy the target cells more efficiently by complement-dependent cytotoxicity (CDC) or antibody-dependent cellular cytotoxicity (ADCC);

2) The human immune system should not recognize the human antibody as foreign, and therefore the antibody response against such an injected antibody should be less than against a totally foreign non-human antibody or a partially foreign chimeric antibody;

3) injected non-human antibodies have been reported to have a half-life in the human circulation much shorter than the half-life of human antibodies. Injected human antibodies will have a half-life essentially identical to naturally occurring human antibodies, allowing smaller and less frequent doses to be given.

The term “fragment” or “fragment thereof” in reference to a hSEZ6 gene or cDNA sequence, refers to a fragment, or sub-region of an hSEZ6 nucleic acid such that said fragment comprises 15 or more nucleotides that are contiguous in the native nucleic acid molecule as shown in SEQ ID NO:1.

The term “fragment” or “fragment thereof” in reference to a hSEZ682 protein or polypeptide sequence, refers to a fragment, or sub-region of an hSEZ682 protein or polypeptide, such that said fragment comprises 5 or more amino acids that are contiguous in the native polypeptide as shown in at least one of SEQ ID NOS:3, 4, 5, 6, 7, 8, 9, 10, and 11.

The term “hybridization” as used herein refers to a process in which a partially or completely single-stranded nucleic acid molecule joins with a complementary strand through nucleotide base pairing. Hybridization can occur under conditions of low, moderate or high stringency, with high stringency preferred. The degree of hybridization depends upon, for example, the degree of homology, the stringency conditions, and the length of hybridizing strands as known in the art.

The term “inhibit” or “inhibiting” includes the generally accepted meaning, which includes prohibiting, preventing, restraining, slowing, stopping, or reversing progression or severity of a disease or condition.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. Ordinarily, an isolated antibody is prepared by at least one purification step. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. An “isolated antibody” is also intended to mean an antibody that is substantially free of other antibodies having different antigenic specificities (e.g., an isolated antibody that specifically binds hSEZ6 substantially free of antibodies that specifically bind antigens other than hSEZ6). An isolated antibody that specifically binds hSEZ6 may bind hSEZ6 molecules from other species (discussed in further detail below)).

The terms “interacting polypeptide segment” and “interacting polypeptide sequence” refer to a portion of a hybrid protein that can form a specific binding interaction with a portion of a second hybrid protein under suitable binding conditions. Generally, a portion of the first hybrid protein preferentially binds to a portion of the second hybrid protein forming a heterodimer or higher order heteromultimer comprising the first and second hybrid proteins; the binding portions of each hybrid protein are termed interacting polypeptide segments. Generally, interacting polypeptides can form heterodimers with a dissociation constant (KD) of at least about 1×10³ M⁻¹, usually at least 1×10⁴ M⁻¹, typically at least 1×10⁵ M⁻¹, preferably at least 1×10⁶ M⁻¹ to 1×10⁷ M⁻¹ or more, under suitable physiological conditions.

By “isolated” nucleic acid molecule(s) is intended a nucleic acid molecules DNA, or RNA, which has been removed from its native or naturally occurring environment. For example, recombinant nucleic acid molecules contained or generated in culture, a vector and/or a host cell are considered isolated for the purposes of the present invention. Further examples of isolated nucleic acid molecules include recombinant nucleic acid molecules maintained in heterologous host cells or purified (partially or substantially) nucleic acid molecules in solution. Isolated RNA molecules include in vivo or in vitro RNA transcripts of the nucleic acid molecules of the present invention. Isolated nucleic acid molecules according to the present invention further include such molecules produced synthetically, purified from or provided in cells containing such nucleic acids, where the nucleic acid exists in other than a naturally occurring form, quantitatively or qualitatively. “Isolated” used in reference to at least one polypeptide of the invention describes a state of isolation such that the peptide or polypeptide is not in a naturally occurring form and/or has been purified to remove at least some portion of cellular or non-cellular molecules with which the protein is naturally associated. However, “isolated” may include the addition of other functional or structural polypeptides for a specific purpose, where the other peptide may occur naturally associated with at least one polypeptide of the present invention, but for which the resulting compound or composition does not exist naturally.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally can not be predicted with complete accuracy.

Methods for predicting whether a protein has a SP sequence, as well as the cleavage point for that sequence, are available. Analysis of the amino acid sequence of the proteins described herein indicated the cleavage point is amino acid 14 and amino acid 25, preferably after amino acid 17 but before amino acid 25, more preferably after amino acid 20 but before amino acid 25, and most preferably after amino acid 24 and before amino acid 25 as presented in SEQ ID NO:3. The resulting mature protein is represented in one non-limiting example by SEQ ID NO: 4. As one of ordinary skill would appreciate, however, cleavage sites sometimes vary from organism to organism and cannot be predicted with absolute certainty. Accordingly, the present invention provides polypeptides having a sequence of 90-100% of the contiguous sequence shown in SEQ ID NO: 3 which have an N-terminus beginning within 10 residues (i.e., +or −10 residues) of the predicted cleavage point prior to amino acid 25 of SEQ ID NO:3. However, cleavage sites for a secreted protein may be determined experimentally by amino-terminal sequencing of the one or more species of mature proteins found within a purified preparation of the protein.

The term “multimer” comprises dimer and higher order complexes (trimer, tetramer, pentamer, hexamer, heptamer, octamer, etc.). “Homomultimer” refers to complexes comprised of the same subunit species. “Heteromultimer” refers to complexes comprised or more than one subunit species.

A “nucleic acid probe,” “oligonucleotide probe,” or “probe” as used herein comprises at least one detectably labeled or unlabeled nucleic acid which hybridizes under specified hybridization conditions with at least one other nucleic acid. This term also refers to a single- or partially double-stranded nucleic acid, oligonucleotide or polynucleotide that will associate with a complementary or partially complementary target nucleic acid to form at least a partially double-stranded nucleic acid molecule. A nucleic acid probe may be an oligonucleotide or a nucleotide polymer. A probe can optionally contain a detectable moiety which may be attached to the end(s) of the probe or be internal to the sequence of the probe, termed a “detectable probe” or “detectable nucleic acid probe.” Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “polynucleotide” comprises at least 10-20 nucleotides of a nucleic acid (RNA, DNA or combination thereof), provided by any means, such as synthetic, recombinant isolation or purification method steps.

The term “variant” as used herein in reference to a hSEZ6 polynucleotide or a hSEZ6 polypeptide is intended to encompass the hSEZ682 polynucleotide or hSEZ682 polypeptide as shown in SEQ ID NO:1 or 3, respectively, as well any fragments thereof, that further comprise at least one of the various types of modifications discussed hereinbelow and have at least about 90% amino acid sequence identity with the corresponding non-variant hSEZ6 polypeptide. Such hSEZ6 polypeptide variants include, for instance, hSEZ6 polypeptides wherein one or more amino acid residues are added, substituted or deleted, at the N- or C-terminus or within the sequence of any one of SEQ ID NOS:3-11. Ordinarily, an hSEZ6 polypeptide variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the corresponding amino acid sequence shown in at least one of the sequences as shown in SEQ ID NO:3-11, with or without the signal peptide.

The phrase “percent (%) identity” with respect to the amino acid sequences identified herein is defined as the percentage of amino acid residues in a candidate sequence that are identical with the amino acid residues in an hSEZ6 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the % identity values used herein are generated using WU-BLAST-2 [Altschul, et al., Methods in Enzymology 266: 460-80 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a % amino acid sequence identity value is determined by dividing (a) the number of matching identical amino acid residues between the amino acid sequence of the hSEZ6 polypeptide of interest and the comparison amino acid sequence of interest (i.e., the sequence against which the hSEZ6 polypeptide of interest is being compared) as determined by WU-BLAST-2, by (b) the total number of amino acid residues of the hSEZ6 polypeptide of interest.

“hSEZ6 variant polynucleotide,” “hSEZ6 polynucleotide variant,” or “hSEZ6 variant nucleic acid sequence” is intended to refer to a nucleic acid molecule having at least about 75% nucleic acid sequence identity with the corresponding polynucleotide sequence shown in at least one of the sequences as shown in SEQ ID NOS:1 or 2. Ordinarily, an hSEZ6 polynucleotide variant will have at least about 75% nucleic acid sequence identity, more preferably at least about 80% nucleic acid sequence identity, yet more preferably at least about 81% nucleic acid sequence identity, yet more preferably at least about 82% nucleic acid sequence identity, yet more preferably at least about 83% nucleic acid sequence identity, yet more preferably at least about 84% nucleic acid sequence identity, yet more preferably at least about 85% nucleic acid sequence identity, yet more preferably at least about 86% nucleic acid sequence identity, yet more preferably at least about 87% nucleic acid sequence identity, yet more preferably at least about 88% nucleic acid sequence identity, yet more preferably at least about 89% nucleic acid sequence identity, yet more preferably at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96,% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequences shown above. Variants specifically exclude or do not encompass the native nucleotide sequences shown in SEQ ID NOS:1 and 2.

The phrase “percent (%) nucleic acid sequence identity” or “percent (%) identity” with respect to the hSEZ6 polynucleotide sequences identified herein is defined as the percentage of nucleotides in a candidate sequence that are identical with the nucleotides in the hSEZ6 sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, percent nucleic acid identity values are generated using the WU-BLAST-2 (BlastN module) computer program [Altschul, et al., Methods in Enzymology 266: 460-80 (1996)). Most of the WU-BLAST-2 search parameters are set to the default values. Those not set default values, i.e., the adjustable parameters, are set with the following values: overlap span=1; overlap fraction=0.125; word threshold (T)=11; and scoring matrix=BLOSUM62. For purposes herein, a % nucleic acid sequence identity value is determined by dividing (a) the number of matching identical nucleotides between the nucleic acid sequence of the hSEZ6 polypeptide-encoding nucleic acid molecule of interest and the comparison nucleic acid molecule of interest (i.e., the sequence against which the hSEZ6 polypeptide-encoding nucleic acid molecule of interest is being compared) as determined by WU-BLAST-2, by (b) the total number of nucleotides of the hSEZ6 polypeptide-encoding nucleic acid molecule of interest.

A “primer” is a nucleic acid fragment or oligonucleotide which functions as an initiating substrate for enzymatic or synthetic elongation of, for example, a nucleic acid molecule, e.g., using an amplification reaction, such as, but not limited to, a polymerase chain reaction (PCR), as known in the art.

The term “stringency” refers to hybridization conditions for nucleic acids in solution. High stringency conditions disfavor non-homologous base pairing. Low stringency conditions have much less of this effect. Stringency may be altered, for example, by changes in temperature and/or salt concentration, or other conditions, as well known in the art.

A non-limiting example of “high stringency” conditions includes, for example, (a) a temperature of about 42° C., a formamide concentration of about 20%, and a low salt (SSC) concentration, or, alternatively, a temperature of about 65° C., or less, and a low salt (SSPE) concentration; (b) hybridization in 0.5 M NaHPO4, 7% sodium dodecyl sulfate (SDS), 1 mM EDTA at 65° C. (See, e.g., Ausubel, et al., ed., Current Protocols in Molecular Biology, 1987-1998, Wiley Interscience, New York, at §2.10.3). “SSC” comprises a hybridization and wash solution. A stock 20× SSC solution contains 3 M sodium chloride, 0.3 M sodium citrate, pH 7.0. “SSPE” comprises a hybridization and wash solution. A 1× SSPE solution contains 180 mM NaCl, 9 mM Na₂HPO₄, 0.9 mM NaH₂PO₄ and 1 mM EDTA, pH 7.4.

The terms “treating,” “treatment,” and “therapy” as used herein refer to curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

The term “vector” as used herein refers to a nucleic acid compound used for introducing exogenous or endogenous nucleic acid into host cells. A vector comprises a nucleotide sequence which may encode one or more polypeptide molecules. Plasmids, cosmids, viruses and bacteriophages, in a natural state or which have undergone recombinant engineering, are non-limiting examples of commonly used vectors to provide recombinant vectors comprising at least one desired isolated nucleic acid molecule.

Nucleic Acid Molecules

Using the information provided herein, such as the nucleotide sequences encoding at least 90-100% of the contiguous amino acids of at least one of SEQ ID NOS:3-11, specified fragments or variants thereof, or a deposited vector comprising at least one of these sequences, a nucleic acid molecule of the present invention encoding an hSEZ6 polypeptide can be obtained using well-known methods.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combination thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention include nucleic acid molecules comprising an open reading frame (ORF) shown in at least one of SEQ ID NOS:1, or 2; nucleic acid molecules comprising the coding sequence for an hSEZ6 polypeptide; and nucleic acid molecules which comprise a nucleotide sequence substantially different from those described above but which, due to the degeneracy of the genetic code, still encode at least one hSEZ6 polypeptide as described herein. Of course, the genetic code is well known in the art. Thus, it would be routine for one skilled in the art to generate such degenerate nucleic acid variants-that code for specific hSEZ6 polypeptides of the present invention. See, e.g., Ausubel, et al., supra, and such nucleic acid variants are included in the present invention.

In a further embodiment, nucleic acid molecules are provided encoding the mature hSEZ6 polypeptide or the full-length hSEZ6 polypeptide lacking the N-terminal methionine.

The invention also provides an isolated nucleic acid molecule having the nucleotide sequence shown in at least one of SEQ ID NOS:1, or 2, or a nucleic acid molecule having a sequence complementary thereto. Such isolated molecules, particularly nucleic acid molecules, are useful as probes for gene mapping by in situ hybridization with chromosomes, and for detecting transcription, translation and/or expression of the hSEZ6 gene in human tissue, for instance, by Northern blot analysis for mRNA detection.

Unless otherwise indicated, all nucleotide sequences identified by sequencing a nucleic acid molecule herein can be or were identified using an automated nucleic acid sequencer. All amino acid sequences of polypeptides encoded by nucleic acid molecules identified herein can be or were identified by codon correspondence or by translation of a nucleic acid sequence identified using method steps as described herein or as known in the art. Therefore, as is well known in the art, any nucleic acid sequence identified by this automated approach and identified herein may contain some errors which are reproducibly correctable by re-sequencing based upon an available or a deposited vector or host cell containing the nucleic acid molecule using well-known methods.

Nucleotide sequences identified by automation are typically at least about 95% to at least about 99.999% identical to the actual nucleotide sequence of the sequenced nucleic acid molecule. The actual sequence can be more precisely identified by other approaches including manual nucleic acid sequencing methods well known in the art. As is also known in the art, a single insertion or deletion in an identified nucleotide sequence compared to the actual sequence will cause a frame shift in translation of the nucleotide sequence. As a result of the frame-shift the identified amino acid sequence encoded by an identified nucleotide sequence will be completely different from the amino acid sequence actually encoded by the sequenced nucleic acid molecule, beginning at the point of such an insertion or deletion.

Nucleic Acid Fragments

The present invention is further directed to fragments of the isolated nucleic acid molecules described herein. It is further intended to mean fragments of at least about 15 nucleotides, and at least about 40 nucleotides in length, which are useful, inter alia as diagnostic probes and primers as described herein. Of course, larger fragments such as at least about 50, 100, 120, 200, 500, 1000, 1500, 20.00, 2500, 3000, 3500, and/or 4000 or more nucleotides in length, are also useful according to the present invention as are fragments corresponding to most, if not all, of the nucleotide sequence (or the deposited cDNA) as shown at least one of SEQ ID NOS:1 or 2. By a fragment at least 15 nucleotides in length, for example, is intended fragments which include 15 or more contiguous nucleotides from the nucleotide sequence as shown in at least one of SEQ ID NOS:1 or 2, as determined by methods known in the art (See e.g., Ausubel, supra, Chapter 7).

Such nucleotide fragments are useful according to the present invention for screening DNA sequences that code for one or more fragments of an hSEZ6 polypeptide as described herein. Such screening, as a non-limiting example can include the use of so-called “DNA chips” for screening DNA sequences of the present invention of varying lengths, as described, e.g., in U.S. Pat. Nos. 5,631,734, 5,624,711, 5,744,305, 5,770,456, 5,770,722, 5,675,443, 5,695,940, 5,710,000, 5,733,729, which are entirely incorporated herein by reference.

As indicated, nucleic acid molecules of the present invention can comprise a nucleic acid encoding an hSEZ6 polypeptide and can include, but is not limited to, those encoding the amino acid sequence of the mature polypeptide, by itself. In addition, the present invention includes polynucleotides comprising the coding sequence for the mature polypeptide joined with additional coding sequences, such as the coding sequence of at least one signal leader or fusion peptide.

Also provided by the present invention are nucleic acid molecules encoding the mature hSEZ6 polypeptide, with or without the aforementioned additional coding sequences, together with additional, non-coding sequences, including but not limited to, introns and non-coding 5′ and 3′ sequences, such as any transcribed, non-translated sequences that play a role in transcription, mRNA processing, including splicing and polyadenylation signals (for example, ribosome binding and stability of mRNA). Furthermore, the present invention includes hSEZ6 polynucleotides encoding hSEZ6 polypeptides having additional amino acids which provide additional functionalities. Thus, the sequence encoding a polypeptide can be fused to a marker sequence, such as a sequence encoding a peptide that facilitates purification of the fused polypeptide.

Preferred nucleic acid fragments of the present invention also include nucleic acid molecules encoding epitope-bearing portions of an hSEZ6 polypeptide.

Oligonucleotide and Polynucleotide Probes and/or Primers

In another aspect, the invention provides a polynucleotide (either DNA or RNA) that comprises at least about 15 nt, still more preferably at least about 30 nt, and even more preferably at least about 30-2000 nt of a nucleic acid molecule described herein. These are useful as diagnostic probes and primers as discussed above and in more detail below.

By a portion of a polynucleotide of “at least 15 nt in length,” for example, is intended 15 or more contiguous nucleotides from the nucleotide sequence of the reference polynucleotide (e.g., at least one nucleotide sequence as shown in at least one of SEQ ID NOS:1 or 2).

Of course, a polynucleotide which hybridizes only to a poly-A sequence (such as the 3′ terminal poly(A) of the hSEZ6 cDNA shown as SEQ ID NO:1, or to a complementary stretch of T (or U) resides, would not be included in a probe of the invention, since such a polynucleotide would hybridize to any nucleic acid molecule containing a poly (A) stretch or the complement thereof (e.g., practically any double-stranded cDNA clone).

The present invention also provides subsequences of full-length nucleic acids. Any number of subsequences can be obtained by reference to at least one of SEQ ID NOS:1, 2, or a complementary sequence thereof, and using primers which selectively amplify, under stringent conditions to: at least two sites to the polynucleotides of the present invention, or to two sites within the nucleic acid which flank and comprise a polynucleotide of the present invention, or to a site within a polynucleotide of the present invention and a site within the nucleic acid which comprises it. A variety of methods for obtaining 5′ and/or 3′ ends is well known in the art. See, e.g., RACE (Rapid Amplification of Complementary Ends) as described in M. A. Frohman, PCR Protocols: A Guide to Methods and Applications, M. A. Innis, et al, Eds., Academic Press, Inc., San Diego, Calif., pp. 28-38 (1990); see also, U.S. Pat. No. 5,470,722, and Ausubel, et al., Current Protocols in Molecular Biology, Chapter 15, Eds., John Wiley & Sons, N.Y. (1989-1999). Thus, the present invention provides hSEZ6 polynucleotides having the sequence of the hSEZ6 gene, nuclear transcript, cDNA, or complementary sequences and/or subsequences thereof.

Primer sequences can be obtained by reference to a contiguous subsequence of a polynucleotide of the present invention. Primers are chosen to selectively hybridize, under PCR amplification conditions, to a polynucleotide of the present invention in an amplification mixture comprising a genomic and/or cDNA library from the same species. Generally, the primers are complementary to a subsequence of the amplified nucleic acid. In some embodiments, the primers will be constructed to anneal at their 5′ terminal ends to the codon encoding the carboxy or amino terminal amino acid residue (or the complements thereof) of the polynucleotides of the present invention. The primer length in nucleotides is selected from the group of integers consisting of from at least 15 to 50. Thus, the primers can be at least 15, 18, 20, 25, 30, 40, or 50 nucleotides in length or any range or value therein. A non-annealing sequence at the 5′ end of the primer (a “tail”) can be added, for example, to introduce a cloning site at the terminal ends of the amplified DNA.

The amplification primers may optionally be elongated in the 3′ direction with additional contiguous or complementary nucleotides from the polynucleotide sequences, such as at least one of. SEQ ID NOS:1 or 2, from which they are derived. The number of nucleotides by which the primers can be elongated is selected from the group of integers consisting of from at least 1 to at least 25. Thus, for example, the primers can be elongated with an additional 1, 5, 10, or 15 nucleotides or any range or value therein. Those of skill will recognize that a lengthened primer sequence can be employed to increase specificity of binding (i.e., annealing) to a target sequence, or to add useful sequences, such as links or restriction sites (See e.g., Ausubel, supra, Chapter 15).

The amplification products can be translated using expression systems well known to those of skill in the art and as discussed, infra. The resulting translation products can be confirmed as polypeptides of the present invention by, for example, assaying for the appropriate catalytic activity (e.g., specific activity and/or substrate specificity), or verifying the presence of one or more linear epitopes which are specific to a polypeptide of the present invention. Methods for protein synthesis from PCR derived templates are known in the art (See e.g., Ausubel, supra, Chapters 9, 10, 15; Coligan, Current Protocols in Protein Science, supra, Chapter 5) and available commercially. See, e.g., Amersham Life Sciences, Inc., Catalog '97, p. 354.

Polynucleotides Which Selectively Hybridize to a Polynucleotide as Described Herein

The present invention provides isolated nucleic acids that hybridize under high stringency conditions to a polynucleotide disclosed herein, e.g., SEQ ID NOS:1 or 2. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low stringency hybridization conditions are typically, but not exclusively, employed with sequences having a reduced sequence identity relative to complementary sequences. Moderate and high stringency conditions can optionally be employed for sequences of greater identity, generally having about 80% sequence identity. Low stringency conditions allow selective hybridization of sequences having about 70% sequence identity and can be employed to identify orthologous or paralogous sequences.

Optionally, polynucleotides of this invention will encode an epitope of a polypeptide encoded by the polynucleotides described herein. The polynucleotides of this invention embrace nucleic acid sequences that can be employed for selective hybridization to a polynucleotide encoding a polypeptide of the present invention.

Screening polypeptides for specific binding to antibodies or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256.

See also, U.S. Pat. Nos. 5,658,754; and 5,643,768. Peptide display libraries, vector, and screening kits are commercially available from such suppliers as Invitrogen (Carlsbad, Calif.).

Polynucleotides Complementary to the Polynucleotides

As indicated above, the present invention provides isolated nucleic acids comprising hSEZ6 polynucleotides, wherein the polynucleotides are complementary to the polynucleotides described herein, above. As those of skill in the art will recognize, complementary sequences base pair throughout the entirety of their length with such polynucleotides (i.e., have 100% sequence identity over their entire length). Complementary bases associate through hydrogen bonding in double-stranded nucleic acids. For example, the following base pairs are complementary: guanine and cytosine; adenine and thymine; and adenine and uracil. (See, e.g., Ausubel, supra, Chapter 67; or Sambrook, supra)

Construction of Nucleic Acids

The isolated nucleic acids of the present invention can be made using (a) standard recombinant methods, (b) synthetic techniques, (c) purification techniques, or combinations thereof, as well known in the art.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention.

Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. (See, e.g., Ausubel, supra, Chapters 1-5; or Sambrook, supra)

Recombinant Methods for Constructing Nucleic Acids

The isolated nucleic acid compositions of this invention, such as RNA, cDNA, genomic DNA, or a hybrid thereof, can be obtained from biological sources using any number of cloning methodologies known to those of-skill in the art. In some embodiments, oligonucleotide probes that selectively hybridize, under stringent conditions, to the polynucleotides of the present invention are used to identify the desired sequence in a cDNA or genomic DNA library. While isolation of RNA, and construction of cDNA and genomic libraries is well known to those of ordinary skill in the art. (See, e.g., Ausubel, supra, Chapters 1-7; or Sambrook, supra)

Nucleic Acid Screening and Isolation Methods

A cDNA or genomic library can be screened using a probe based upon the sequence of a polynucleotide of the present invention, such as those disclosed herein. Probes may be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. Temperature, ionic strength, pH and the presence of a partially denaturing solvent such as formamide can control the degree of stringency. Changing the polarity of the reactant solution through, for example, manipulation of the concentration of formamide within the range of 0% to 50% conveniently varies the stringency of hybridization. The degree of complementarity (sequence identity) required for detectable binding will vary in accordance with the stringency of the hybridization medium and/or wash medium. The degree of complementarity will optimally be 100%; however, it should be understood that minor sequence variations in the probes and primers may be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification which uses anti-sense RNA to the target sequence as a template for double-stranded DNA synthesis (U.S. Pat. No. 5,130,238 to Malek, et al, with the tradename NASBA), the entire contents of which are herein incorporated by reference. (See, e.g., Ausubel, supra, Chapter 15; or Sambrook, supra)

For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, Sambrook, and Ausubel (e.g., Chapter 15) supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

Synthetic Methods for Constructing Nucleic Acids

The isolated nucleic acids of the present invention can also be prepared by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Letts. 22:1859-1862 (1981); the solid phase phosphoramidite triester method described by Beaucage and Caruthers, Tetra. Letts. 22(20):1859-1862 (1981), e.g., using an automated synthesizer, e.g., as described in Needham-Van Devanter, et al., Nucleic Acids Res. 12:6159-6168 (1984); and the solid support method of U.S. Pat. No. 4,458,066. Chemical synthesis generally produces a single-stranded oligonucleotide, which may be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences may be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding a full-length polypeptide of the present invention, can be used to construct a recombinant expression cassette which can be introduced into at least one desired host cell. A recombinant expression cassette will typically comprise a polynucleotide of the present invention operably linked to transcriptional initiation regulatory sequences that will direct the transcription of the polynucleotide in the intended host cell.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter hSEZ6 content and/or composition in a desired tissue.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo or in vitro by mutation, deletion and/or substitution.

A polynucleotide of the present invention can be expressed in either sense or anti-sense orientation as desired. It will be appreciated that control of gene expression in either sense or anti-sense orientation can have a direct impact on the observable characteristics.

Another method of suppression is sense suppression. Introduction of nucleic acid configured in the sense orientation has been shown to be an effective means by which to block the transcription of target genes.

A variety of cross-linking agents, alkylating agents and radical generating species as pendant groups on polynucleotides of the present invention can be used to bind, label, detect and/or cleave nucleic acids. Knorre, et al., Biochimie 67:785-789 (1985); Vlassov, et al., Nucleic Acids Res. 14:4065-4076 (1986); Iverson and Dervan, J. Am. Chem. Soc. 109:1241-1243 (1987); Meyer, et al., J. Am. Chem. Soc. 111:8517-8519 (1989); Lee, et al., Biochemistry 27:3197-3203 (1988); Home, et al., J. Am. Chem. Soc. 112:2435-2437 (1990); Webb and Matteucci, J. Am. Chem. Soc. 108:2764-2765 (1986); Nucleic Acids Res. 14:7661-7674 (1986); Feteritz, et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681,941, each entirely incorporated herein by reference.

Vectors and Host Cells

The present invention also relates to vectors that include isolated nucleic acid molecules of the present invention, host cells that are genetically engineered with the recombinant vectors, and the production of hSEZ6 polypeptides or fragments thereof by recombinant techniques, as is well known in the art. See, e.g., Sambrook, et al., supra; Ausubel, supra, Chapters 1-9, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, or any other suitable promoter. The skilled artisan will know other suitable promoters. The expression constructs will further contain sites for transcription initiation, termination and, in the transcribed region, a ribosome-binding site for translation. The coding portion of the mature transcripts expressed by the constructs will preferably include a translation initiating at the beginning and a termination codon (e.g., UAA, UGA or UAG) appropriately positioned at the end of the mRNA to be translated, with VAA and VAG preferred for mammalian or eukaryotic cell expression.

Expression vectors will preferably include at least one selectable marker. Such markers include, e.g., dihydrofolate reductase, ampicillin (G418), hygromycin or neomycin resistance for eukaryotic cell culture, and tetracycline or ampicillin resistance genes for culturing in E. coli and other bacteria or prokaryotics. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art. Vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Preferred eucaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan. See, e.g., Ausubel, supra, Chapter 1; Coligan, Current Protocols in Protein Science, supra, Chapter 5.

Introduction of a vector construct into a host cell can be effected by calcium phosphate transfection, DEAE-dextran mediated transfection, cationic lipid-mediated transfection, electroporation, transduction, infection or other methods. Such methods are described in many standard laboratory manuals, such as Sambrook, supra, Chapters 1-4 and 16-18; Ausubel, supra, Chapters 1, 9, 13, 15, 16.

Polypeptide(s) of the present invention can be expressed in a modified form, such as a fusion protein, and can include not only secretion signals, but also additional heterologous functional regions. For instance, a region of additional amino acids, particularly charged amino acids, can be added to the N-terminus of a polypeptide to improve stability and persistence in the host cell, during purification, or during subsequent handling and storage. Also, peptide moieties can be added to a polypeptide to facilitate purification. Such regions can be removed prior to final preparation of a polypeptide. Such methods are described in many standard laboratory manuals, such as Sambrook, supra, Chapters 17 and 18; Ausubel, supra, Chapters 16, 17 and 18.

Expression of Proteins in Host Cells

Using nucleic acids of the present invention, one may express a protein of the present invention in a recombinantly engineered cell, such as bacteria, yeast, insect, or mammalian cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible) followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Alternatively, nucleic acids of the present invention can be expressed in a host cell by turning on (by manipulation) in a host cell that contains endogenous DNA encoding a polypeptide of the present invention. Such methods are well known in the art, e.g., as described in U.S. Pat. Nos. 5,580,734, 5,641,670, 5,733,746, and 5,733,761, entirely incorporated herein by reference.

Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., Nature 198:1056 (1977)), the tryptophan (trp) promoter system (Goeddel, et al., Nucleic Acids Res. 8:4057 (1980)) and the lambda derived P L promoter and N-gene ribosome binding site (Shimatake, et al., Nature 292:128 (1981)). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transformed with the plasmid vector DNA. Expression systems for expressing a protein of the present invention are available using Bacillus sp. and Salmonella (Palva, et al., Gene 22:229-235 (1983); Mosbach, et al., Nature 302:543-545 (1983)). See, e.g., Ausubel, supra, Chapters 1-3, 16(Sec.1); and Coligan, supra, Current Protocols in Protein Science, Units 5.1, 6.1-6.7.

Expression in Eukaryotes

A variety of eukaryotic expression systems such as yeast, insect cell lines, plant and mammalian cells, are known to those of skill in the art. As explained briefly below, a nucleic acid of the present invention can be expressed in these eukaryotic systems.

Synthesis of heterologous proteins in yeast is well known. F. Sherman, et al., Methods in Yeast Genetics, Cold Spring Harbor Laboratory (1982) is a well-recognized work describing the various methods available to produce the protein in yeast. Two widely utilized yeast for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen).

Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Illustrative of cell cultures useful for the production of the peptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., Immunol. Rev. 89:49 (1986)), and processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7th edition, 1992).

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See Schneider, J. Embryol. Exp. Morphol. 27:353-365 (1987).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., J. Virol. 45:773-781 (1983)). Additionally, gene sequences to control replication in the host cell may be incorporated into the vector such as those found in bovine papilloma virus type-vectors. (M. Saveria-Campo, Bovine Papilloma Virus DNA, a Eukaryotic Cloning Vector in DNA Cloning Vol. II, a Practical Approach, D. M. Glover, Ed., IRL Press, Arlington, Va., pp. 213-238 (1985)).

Protein Purification

An hSEZ6 polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eucaryotic host, including, for example, bacterial, yeast, higher plant, insect and mammalian cells. Depending upon the host employed in a recombinant production procedure, the polypeptides of the present invention can be glycosylated or can be non-glycosylated. In addition, polypeptides of the invention can also include an initial modified methionine residue, in some cases as a result of host-mediated processes. Such methods are described in many standard laboratory manuals, such as Sambrook, supra, Chapters 17.37-17.42; Ausubel, supra, Chapters 10, 12, 13, 16, 18 and 20.

hSEZ6 Polypeptides and Fragments and/or Variants Thereof

The isolated proteins of the present invention comprise a polypeptide as well as fragments and/or variants thereof encoded by any one of the polynucleotides of the present invention as discussed more fully, supra.

The proteins of the present invention can comprise any number of contiguous amino acid residues from the polypeptide as shown in SEQ ID NO:3. Exemplary polypeptide sequences are provided in SEQ ID NOS:3-11.

Generally, the polypeptides of the present invention will, when presented as an immunogen, elicit production of an antibody specifically reactive to a polypeptide of the present invention encoded by a polynucleotide of the present invention as described, supra. Exemplary polypeptides include those which are full-length, such as those disclosed herein. Further, the proteins of the present invention will not bind to antisera raised against a polypeptide of the present invention which has been fully immunosorbed with the same polypeptide. Immunoassays for determining binding are well known to those of skill in the art. A preferred immunoassay is a competitive immunoassay as discussed, infra. Thus, the proteins of the present invention can be employed as immunogens for constructing antibodies immunoreactive to a protein of the present invention for such exemplary utilities as immunoassays or protein purification techniques.

An hSEZ6 polypeptide of the present invention can include one or more amino acid substitutions, deletions or additions, either from natural mutations or human manipulation, as specified herein. Of course, the number of amino acid substitutions a skilled artisan would make depends on many factors, including those described above. Generally speaking, the number of amino acid substitutions, insertions or deletions for any given hSEZ6 polypeptide may be anywhere from 2-100.

Amino acids in an hSEZ6 polypeptide of the present invention that are essential for protein-protein binding or ligand-protein binding can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity. Sites that are critical for protein-protein binding or ligand-protein binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

A hSEZ6 polypeptide of the present invention can include but is not limited to the mature protein form of the polypeptide as shown in SEQ ID NO:4.

An hSEZ6 polypeptide can further comprise a polypeptide of 853 or 829 contiguous amino acids as shown in SEQ ID NOS:3 or 4, respectively.

An hSEZ6 polypeptide includes any amino acid sequence selected from the group of sequences as shown in SEQ ID NOS:3, 4, 5, 6, 7, 8, 9, 10, 11, as well as fragments thereof. Additionally, the present invention encompasses hSEZ6 polypeptide variants comprising any of the above mentioned hSEZ6 polypeptides wherein said polypeptide further comprises at least one mutation. Variations in the full-length sequence hSEZ6 or in various domains of the hSEZ6 polypeptide described herein can be made, for example, using any of the techniques and guidelines for conservative and non-conservative mutations set forth, for instance, in U.S. Pat. No. 5,364,934. Variations may be a substitution, deletion or insertion of one or more codons encoding hSEZ6 polypeptide that results in a change in the amino acid sequence of the hSEZ6 polypeptide as compared with the native sequence hSEZ6 polypeptide or an hSEZ6 polypeptide as disclosed herein. Optionally, the variation is by substitution of at least one amino acid with any other amino acid in one or more of the domains of the hSEZ6 polypeptide. Guidance in determining which amino acid residue may be inserted, substituted or deleted without adversely affecting the desired activity may be found by comparing the sequence of the hSEZ6 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity (such as in any of the in vitro assays described herein) for activity exhibited by the full-length or mature native polypeptide sequence. Preferred hSEZ6 variants include those having at least one substitution, or deletion of at least one amino acid residue selected from the group consisting of 26I, 27T, 29E, 31H, 33T, 36R, 51S, 52D, 83R, 85E, 87A, 88P, 89Q, 98A, 11T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L,208V, 209E, 213I, 214T, 217G, 235V, 240P, 260A, 261P, 265S, 273Y, 288E, 293Q, 298I, 339L, 380H, 394F, 408Q, 449P, 452S, 477N, 491E, 503R, 509F, 530R, 546A, 548S, 577H, 642S, 667G 690A, 708N, 722N, 749I, 757S, 798V, 806T, 809A, and 835F of SEQ ID NO:3 or the corresponding amino acids of SEQ ID NOS: 4-11.

Human hSEZ6 polypeptide fragments are also provided herein. Such fragments may be truncated at the N-terminus or C-terminus, or may lack internal residues, for example, when compared with the hSEZ6 polypeptides as shown in SEQ ID NO:3, 4, 5, 6, 7, 8, 9, 10, and 11. Certain fragments contemplated by the present invention may lack amino acid residues that are not essential for a desired biological activity of the hSEZ6 polypeptide.

Human hSEZ6 polypeptide fragments may be prepared by any of a number of conventional techniques. Desired peptide fragments may be chemically synthesized. An alternative approach involves generating hSEZ6 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment by polymerase chain reaction (PCR). Oligonucleotides that define the desired termini of the DNA fragment are employed at the 5′ and 3′ primers in the PCR. Preferably, hSEZ6 polypeptide fragments share at least one biological and/or immunological activity with at least one of the hSEZ6 polypeptides as shown in SEQ ID NO:3, 4, 5, 6, 7, 8, 9, 10, and 11.

Also, any one of the hSEZ6 polypeptides, hSEZ6 polypeptide fragments, and/or hSEZ6 polypeptide variants disclosed herein may contain one or more of the many possible types of polypeptide modifications known in the art. For example, contemplatable hSEZ6 polypeptides include, but are not limited to, branched polypeptides, as a result of ubiquitination, and cyclic polypeptides, with or without branching. Cyclic, branched, and branched cyclic hSEZ6 polypeptides may result from post-translation natural processes or may be made by synthetic methods. Contemplated modifications also nclude acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Creighton, Proteins—Structure and Molecular Properties, 2nd Ed., W. H. Freeman and Company, New York (1993); Johnson, Post-translational Covalent Modification of Proteins, Academic Press, New York, pp. 1-12 (1983); Seifter, et al., Meth. Enzymol. 182: 626-46 (1990); Rattan, et al., Ann. NY Acad. Sci. 663: 48-62 (1992).

Covalent modifications of hSEZ6 polypeptides are also included within the scope of this invention. One type of covalent modification includes reacting targeted amino acid residues of an hSEZ6 polypeptide with an organic derivatizing agent that is capable of reacting with selected side chains or the N- or C-terminal residues of an hSEZ6 polypeptide. Derivatization with bifunctional agents is useful, for instance, for crosslinking hSEZ6 to a water-insoluble support matrix or surface for use in the method for purifying anti-hSEZ6 polypeptide antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis-(succinimidylpropionate), bifunctional maleimides such as bis-N-maleimido-1,8-octane and agents such as methyl-3-((p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the alpha-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the hSEZ6 polypeptides comprises altering the native glycosylation pattern of the polypeptide. “Altering the native glycosylation pattern” is intended for purposes herein to mean deleting one or more carbohydrate moieties found in native sequence hSEZ6 polypeptide and/or adding one or more glycosylation sites that are not present in the native sequence hSEZ6 polypeptide. Additionally, the phrase includes qualitative changes in the glycosylation of the native proteins, involving a change in the nature and proportions of the various carbohydrate moieties present.

Addition of glycosylation sites to hSEZ6 polypeptides may be accomplished by altering the amino acid sequence thereof. The alteration may be made, for example, by the addition of or substitution by one or more serine or threonine residues to the native sequence hSEZ6 polypeptide (for O-linked glycosylation sites). The hSEZ6 amino acid sequences may optionally be altered through changes at the DNA level, particularly by mutating the DNA encoding the hSEZ6 polypeptides at preselected bases such that codons are generated that will translate into the desired amino acids.

Another means of increasing the number of carbohydrate moieties on the hSEZ6 polypeptides is by chemical or enzymatic coupling of glycosides to the polypeptide. Such methods are described in the art, e.g., in WO 87/05330, published Sep. 11, 1987, and in Aplin and Wriston, CRC Crit. Rev. Biochem., pp. 259-306 (1981).

Removal of carbohydrate moieties present on the hSEZ6 polypeptide may be accomplished chemically or enzymatically or by mutational substitution of codons encoding for amino acid residues that serve as targets for glycosylation. Chemical deglycosylation techniques are known in the art and described, for instance, by Sojar, et al., Arch. Biochem. Biophys. 259: 52-7 (1987) and by Edge, et al., Anal. Biochem. 118: 131-7 (1981). Enzymatic cleavage of carbohydrate moieties on polypeptides can be achieved by the use of a variety of endo- and exo-glycosidases as described by Thotakura, et al., Meth. Enzymol. 138: 350-9 (1987).

Another type of covalent modification of hSEZ6 comprises linking any one of the hSEZ6 polypeptides to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

Also contemplated by the present invention are hSEZ6 polypeptide variants that comprise one or more substitutions, deletions, insertions, or changes in glycosylation sites or patterns as compared to the hSEZ6 polypeptides disclosed herein. Such modifications may be directed at improving upon the therapeutic character of the native hSEZ6 polypeptide by increasing that molecule's target specificity, solubility, stability, serum half-life, affinity for targeted receptors, susceptibility to proteolysis, resistance to proteolysis, ease of purification, and/or decreasing the antigenicity and/or required frequency of administration of a hSEZ6 polypeptide. To the extent that any such modifications can be made while substantially retaining the activity and pharmaceutically desirable properties of the hSEZ6 polypeptides or fragments and/or variants thereof are included within the scope of the present invention. The utility of such additionally modified hSEZ6 variants can be determined without undue experimentation by, for example, the methods described herein.

Antigenic/Epitope Comprising hSEZ6 Peptide and Polypeptides

In another aspect the invention provides a peptide or polypeptide comprising an epitope-bearing portion of a polypeptide of the invention according to methods well known in the art. See, e.g., Cooligan, ed., Current Protocols in Immunology, Greene Publishing, NY (1993-1998), Ausubel, supra, each entirely incorporated herein by reference.

The epitope of this polypeptide portion is an immunogenic or antigenic epitope of a polypeptide described herein. An “immunogenic epitope” can be defined as a part of a polypeptide that elicits an antibody response when the whole polypeptide is the immunogen. On the other hand, a region of a polypeptide molecule to which an antibody can bind is defined as an “antigenic epitope.” The number of immunogenic epitopes of a polypeptide generally is less than the number of antigenic epitopes. See, for instance, Geysen, et al., Proc. Natl. Acad. Sci. USA 81:3998-4002 (1983).

As to the selection of peptides or polypeptides bearing an antigenic epitope (i.e., that contain at least a portion of a region of a polypeptide molecule to which an antibody can bind), it is well known in the art that relatively short synthetic peptides that mimic part of a polypeptide sequence are routinely capable of eliciting an antiserum that reacts with the partially mimicked polypeptide. See, for instance, J. G. Sutcliffe, et al., “Antibodies that react with preidentified sites on polypeptides,” Science 219:660-666 (1983).

Antigenic epitope-bearing peptides and polypeptides of the invention are useful to raise antibodies, including monoclonal antibodies, or screen antibodies, including fragments or single chain antibodies, that bind specifically to a polypeptide of the invention. See, e.g., Wilson, et al., Cell 37:767-778 (1984) at 777. Antigenic epitope-bearing peptides and polypeptides of the invention preferably contain a sequence of at least five, more preferably at least nine, and most preferably between at least about 15 to about 30 amino acids contained within the amino acid sequence of a polypeptide of the invention.

The epitope-bearing peptides and polypeptides of the invention can be produced by any conventional means. R. A. Houghten, “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten, et al. (1986).

As one of skill in the art will appreciate, hSEZ6 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker, et al., Nature 331:84-86 (1988)).

Fusion proteins that have a disulfide-linked dimeric structure due to the IgG part can also be more efficient in binding and neutralizing other molecules than the monomeric hSEZ6 polypeptide or polypeptide fragment alone (Fountoulakis, et al., J. Biochem. 270:3958-3964 (1995)).

Production of Antibodies

The polypeptides of this invention and fragments thereof may be used in the production of antibodies. The term “antibody” as used herein describes antibodies, fragments of antibodies (such as, but not limited, to Fab, Fab′, Fab2′, and Fv fragments), and modified versions thereof, as well known in the art (e.g., chimeric, humanized, recombinant, veneered, resurfaced or CDR-grafted). The term “antibody” is meant to include polyclonal antibodies, monoclonal antibodies (MAbs), chimeric antibodies, single-chain polypeptide binding molecules, and anti-idiotypic (anti-id) antibodies. Polyclonal antibodies are heterogeneous populations of antibody molecules derived from the sera of animals immunised with an antigen while monoclonal antibodies (MAbs) are a substantially homogeneous population of antibodies to specific antigens. Polyclonal and MAbs may be obtained by methods known to those skilled in the art (for MAbs, see, for example, Kohler et al., Nature 256:495-497 (1975), Colligan, supra., and U.S. Pat. No. 4,376,110).

Single chain antibodies and libraries thereof are yet another variety of genetically engineered antibody technology that is well known in the art. (See, e.g., R. E. Bird, et al., Science 242:423-426 (1988); PCT Publication Nos. WO 88/01649, WO 90/14430, and WO 91/10737. Single chain antibody technology involves covalently joining the binding regions of heavy and light chains to generate a single polypeptide chain. The binding specificity of the intact antibody molecule is thereby reproduced on a single polypeptide chain.

MAbs may be of any immuno-globulin class including IgG, IgM, IgE, IgA, IgD and any subclass thereof. The hybridoma producing the MAbs of this invention may be cultivated in vitro or in vivo. Production of high titers of MAbs in vivo makes this the presently preferred method of production. Briefly, cells from the individual hybridomas are injected intraperitoneally into pristane-primed BALB/C mice to produce ascites fluid containing high concentrations of the desired MAbs. MAbs of isotype IgM or IgG may be purified from such ascites fluids, or from culture supernatants, using column chromatography methods well known to those of skill in the art.

Chimeric antibodies are molecules in which different portions are derived from different animal species, such as those having variable region derived from a murine MAb and a human immunoglobulin constant region. Chimeric antibodies and methods for their production are known in the art (Cabilly et al., Proc. Natl. Acad. Sci. (USA) 71:3273-3277 (1984); Morrison et al., Proc. Natl. Acad. Sci. (USA) 81:6851-6855 (1984); Boulianne et al., Nature 312:643646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 86/01533 (published Mar. 13, 1986); Kudo et al., European Patent Application 184187 (published Jun. 11, 1986); Sahagan et al., J. Immunol. 137:1066-1074 (1986); Robinson et al., International Patent Publication #PCT/US86/02269 (published May 7, 1987); Liu et al., Proc. Natl. Acad. Sci. (USA) 84:3439-3443 (1987); Sun et al., Proc. Natl. Acad. Sci. (USA) 84:214-218 (1987); Better et al., Science 140:1041-1043 (1988)). These documents are hereby incorporated by reference.

The most preferred method of generating MAbs to the polypeptides and glycopeptides of the present invention comprises producing said MAbs in a transgenic mammal modified in such a way that they are capable of producing fully humanized MAbs upon antigenic challenge. Fully humanized MAbs and methods for their production are generally known in the art (PCT/WO9634096, PCT/WO9633735, and PCT/WO9824893). These documents are hereby incorporated by reference.

An anti-idiotypic (anti-Id) antibody is an antibody which recognizes unique determinants generally associated with the antigen-binding site of an antibody. An anti-Id antibody can be prepared by immunizing an animal of the same species and genetic type (e.g. mouse strain) as the source of the MAb with the MAb to which an anti-Id is being prepared. The immunized animal will recognize and respond to the idiotypic determinants of the immunizing antibody by producing an antibody to these idiotypic determinants (the anti-Id antibody). The anti-Id antibody may also be used as an “immunogen” to induce an immune response in yet another animal, producing a so-called anti-anti-Id antibody. The anti-anti-Id may be epitopically identical to the original MAb which induced the anti-Id. Thus, by using antibodies to the idiotypic determinants of a MAb, it is possible to identify other clones expressing antibodies of identical specificity. Accordingly, MAbs generated against a hSEZ6 protein or glycoprotein of the present invention may be used to induce anti-Id antibodies in suitable animals, such as BALB/C mice and/or any transgenically altered mouse capable of producing fully humanized MAbs. Spleen cells from such immunized mice are used produce anti-Id hybridomas secreting anti-Id MAbs. Further, the anti-Id MAbs can be coupled to a carrier such as keyhole limpet hemocyanin (KLH) and used to immunize additional similar mice. Sera from these mice will contain anti-anti-Id antibodies that have the binding properties of the original MAb specific for a hSEZ6 epitope. The anti-Id MAbs thus have their own idiotypic epitopes, or “idiotopes” structurally similar to the epitope being evaluated, such as a hSEZ6 protein or glycoprotein.

The term “antibody” is also meant to include both intact molecules as well as fragments thereof, such as, for example, Fab and F(ab′), which are capable of binding antigen. Fab and F(ab′), fragments lack the Fc fragment of intact antibody, clear more rapidly from the circulation, and may have less non-specific tissue binding than an intact antibody (Wahl et al., J. Nucl. Med. 24:316-325 (1983)).

It will be appreciated that Fab and F(ab′)a and other fragments of the antibodies useful in the present invention may be used for the detection and quantitation of a hSEZ6. protein or glycoprotein according to methods disclosed herein for intact antibody molecules. Such fragments are typeically produced by proteolytic cleavage, using enzymes such as papain (to produce Fab fragments) or pepsin (to produce F(ab′)₂ fragments).

A polypeptide used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma or other suitable known cells, and allowed to become monoclonal antibody producing hybridoma cells in the manner known to the skilled artisan. Hybridomas that secrete a desired antibody molecule can be screened by a variety of well known methods, for example ELISA assay, Western blot analysis, or radioimmunoassay (Lutz, et al. Exp. Cell Res. 175:109-124 (1988); Monoclonal Antibodies: Principles & Applications, Ed. J. R. Birch & E. S. Lennox, Wiley-Liss (1995); Colligan, supra).

Antibodies included in this invention are useful in diagnostics, therapeutics or in diagnostic/therapeutic combinations. Thus, polypeptides of this invention or suitable fragments thereof can be used to generate polyclonal or monoclonal antibodies, and various inter-species hybrids, or humanized antibodies, or antibody fragments, or single-chain antibodies.

In one aspect, the present invention relates to a method for detecting the presence of or measuring the quantity of a hSEZ6 protein or glycoprotein in a cell, comprising:

(a) contacting said cell or an extract thereof with an antibody specific for an epitope of a hSEZ6 protein or glycoprotein; and

(b) detecting the binding of said antibody to said cell or extract thereof, or measuring the quantity of antibody bound, thereby determining the presence of or measuring the quantity of said hSEZ6 protein or glycoprotein.

For some applications labeled antibodies are desirable. Procedures for labeling antibody molecules are widely known, including for example, the use of radioisotopes, affinity labels, such as biotin or avidin, enzymatic labels, for example horseradish peroxidase, and fluorescent labels, such as FITC or rhodamine (see, e.g., Colligan, supra). Labeled antibodies are useful for a variety of diagnostic applications. In one embodiment the present invention relates to the use of labeled antibodies to detect the presence of an hSEZ6 polypeptide. Alternatively, the antibodies could be used in a screen to identify potential modulators of an hSEZ6 polypeptide. For example, in a competitive displacement assay, the antibody or compound to be tested is labeled by any suitable method. Competitive displacement of an antibody from an antibody-antigen complex by a test compound such that a test compound-antigen complex is formed provides a method for identifying compounds that bind hSEZ6.

Identification of SEZ6-Associating Polypeptides

Proteins that bind to hSEZ6 and/or a complex comprising hSEZ6 are potentially important neuronal regulatory or neuronal developmental proteins. Such proteins may function as novel neuronal chemoattractant or chemorepulsant molecules, neuronal growth factors, and the like. These proteins are referred to herein as hSEZ6 associating proteins. Associating proteins may be isolated by various methods known in the art. Accordingly, the present invention also provides methods for identifying polypeptides which bind to a hSEZ6 polypeptide.

A preferred method of isolating associating proteins is by contacting a hSEZ6 polypeptide to an antibody that binds the hSEZ6 polypeptide, and isolating resultant immune complexes. These immune complexes may contain associating proteins bound to the hSEZ6 polypeptide. The associating proteins may be identified and isolated by denaturing the immune complexes with a denaturing agent and, preferably, a reducing agent. The denatured, and preferably reduced, proteins can be separated on a polyacrylamide gel. Putative associating proteins are then identified on the polyacrylamide gel by one or more of various well known methods (e.g., Coomassie staining, Western blotting, silver staining, etc.) and isolated by resection of a portion of the polyacrylamide gel containing the relevant identified polypeptide and elution of the polypeptide from the gel portion.

A putative associating protein may be identified as an associating protein by demonstration that the protein binds to hSEZ6 and/or a complex comprising hSEZ6. Such binding may be shown in vitro by various means, including, but not limited to, binding assays employing a putative associating protein that has been renatured subsequent to isolation by a polyacrylamide gel electrophoresis method. Alternatively, binding assays employing recombinant or chemically synthesized putative associating protein may be used. For example, a putative associating protein may be isolated and all or part of its amino acid sequence determined by chemical sequencing, such as Edman degradation. The amino acid sequence information may be used to chemically synthesize the putative associating protein or to produce a recombinant putative hSEZ6 associating protein.

hSEZ6 associating proteins may also be identified by cross-linking in vivo with bi-functional cross-linking reagents (e.g., dimethylsuberimidate, glutaraldehyde, etc.) and subsequent isolation of cross-linked products that include a hSEZ6 polypeptide. (For a general discussion of cross-linking, see Kunkel, et al, Mol. Cell. Biochem, 34:3 (1981), which is incorporated herein by reference). Preferably, the bi-functional cross-linking reagent will produce cross-links that may be reversed under specific conditions after isolation of the cross-linked complex so as to facilitate isolation of the associating protein from the Lyar polypeptide. Isolation of cross-linked complexes that include a hSEZ6 polypeptide is preferably accomplished by binding an antibody that binds an hSEZ6 polypeptide with an affinity of at least 1×10⁷ M⁻¹ to a population of cross-linked complexes and recovering only those complexes that bind to the antibody with an affinity of at least 1×10⁷ M⁻¹. Polypeptides that are cross-linked to a hSEZ6 polypeptide are identified as hSEZ6 associating proteins.

Also, an expression library, such as a λgt11 cDNA expression library (Dunn, et al., J. Biol. Chem. 264: 13057 (1989)), can be screened with a labeled hSEZ6 polypeptide to identify cDNAs encoding polypeptides which specifically bind to the hSEZ6 polypeptide. For these procedures, cDNA expression libraries usually comprise mammalian cDNA populations, typically human, mouse, or rat, and may represent cDNA produced from RNA of one cell type, tissue, or organ and one or more developmental stage. Specific binding for screening cDNA expression libraries is usually provided by including one or more blocking agent (e.g., albumin, nonfat dry milk solids, etc.) prior to and/or concomitant with contacting the labeled hSEZ6 polypeptide (and/or labeled anti-hSEZ6 antibody).

Another approach to identifying polypeptide sequences which bind to a predetermined polypeptide sequence (i.e., hSEZ6) has been to use a variation on the so-called “two-hybrid” system. Two-hybrid methods generally rely upon a positive association between two fusion proteins thereby reconstituting a functional transcriptional activator which then induces transcription of a reporter gene operably linked to an appropriate transcriptional activator binding site. Transcriptional activators are proteins that positively regulate the expression of specific genes. They can be functionally dissected into two structural domains: one region that binds to specific DNA sequences and thereby confers specificity, and another region termed the activation domain that binds to protein components of the basal gene expression machinery (Ma and Ptashne, Cell, 55: 443 (1988)). These two domains need to be physically connected in order to function as a transcriptional activator. Two-hybrid systems exploit this requirement by hooking up an isolated DNA binding domain to one protein (protein X), while hooking up the isolated activation domain to another protein (protein Y). When X and Y interact to a significant extent, the DNA binding and activation domains will now be connected and the transcriptional activator function reconstituted. The host strain is engineered so that the reconstituted transcriptional activator drives the expression of a specific reporter gene, which provides the read-out for the protein-protein interaction (Field and Song, (1989); Chein et al., (1991)). Transcription of the reporter gene produces a positive readout, typically manifested either (1) as an enzyme activity (e.g., β-galactosidase) that can be identified by a calorimetric enzyme assay or (2) as enhanced cell growth on a defined medium (e.g., HIS3). A positive readout condition is generally identified as, but not limited to, one or more of the following detectable conditions: (1) an increased transcription rate of a predetermined reporter gene, (2) an increased concentration or abundance of a polypeptide product encoded by a predetermined reporter gene, typically an enzyme which can be readily assayed in vivo, and/or (3) a selectable or otherwise identifiable phenotypic change in a organism harboring the two-hybrid system. Generally, a selectable or otherwise identifiable phenotypic change that characterizes a positive readout condition confers upon the organism (e.g., yeast, bacteria, mammalian cell) either: a selective growth advantage on a-defined medium, a mating phenotype, a characteristic morphology or developmental stage, drug resistance, or a detectable enzymatic activity (e.g., β-galactosidase, luciferase, alkaline phosphatase).

One advantage of a two-hybrid system for monitoring protein-protein interactions is their sensitivity in detection of physically weak, but physiologically important, protein-protein interactions. As such it offers a significant advantage over other methods for detecting protein-protein interactions (e.g., ELISA assay).

Typically, the two-hybrid method is used to identify novel polypeptide sequences which interact with a known protein (Silver S. C. and Hunt S. W., Mol. Biol. Rep., 17:155 (1993); Durfee et al., Genes Devel., 7;555 (1993); Yang et al., Science, 257:680 (1992); Luban et al., Cell 73:1067 (1993); Hardy et al., Genes Devel., 6:801 (1992); Bartel et al., Biotechniques, 14:920 (1993); and VojTek et al., Cell 74:205 (1993)). However, two hybrid systems have also been used to identify interacting structural domains of known proteins (Bardwell et al., Med. Microbio., 8:1177 (1993); Chakraborty et al., J. Biol. Chem., 267:17498 (1992); Staudinger et al., J. Biol. Chem., 268:4608 (1993); and Milne, G. T. and Weaver, D. T. Genes Devel. 7:1755 (1993)) or domains responsible for oligomerization of a single protein (Iwabuchi et al., Oncogene, 8:1693 (1993); Bogerd et al., J. Virol., 67:5030 (1993)).

A preferred two-hybrid system identifies protein-protein interactions in vivo through reconstitution of a transcriptional activator, the yeast Gal4 transcription protein (Fields and Song, Nature, (1989)). The yeast Gal4 protein consists of separable domains responsible for DNA-binding and transcriptional activation. Polynucleotides encoding two hybrid proteins, one consisting of the yeast Gal4 DNA-binding domain fused to a polypeptide sequence of a known protein and the other consisting of the Gal4 activation domain fused to a polypeptide sequence of a second protein, are constructed and introduced into a yeast host cell. Intermolecular binding between the two fusion proteins reconstitutes the Gal4 DNA-binding domain with the Gal4 activation domain, which leads to the transcriptional activation of a reporter gene (e.g., lacZ, HIS3) which is operably linked to a Gal4 binding site.

Alternatively, an E. coli/BCCP interactive screening system or other variations on the two-hybrid system known in the art can be used to identify interacting protein sequences. (See, e.g., Germino et al., Proc. Natl. Acad. Sci. (USA), 90:933 (1993); Guarente, L., Proc. Natl. Acad. Sci. (USA), 90:1639 (1993); Frederickson, R. M., Current Opinion in Biotechnology, 9(1):90-6 (1998); Vidal, M. and Legrain P. Nucleic Acids Research, 27(4):919-29 (1999); Drees, B. L. Current Opinion in Chemical Biology, 3(1):64-70 (1999); Sorimachi H., et al., Protein, Nucleic Acid, Enzyme, 42(14 Suppl):2433-40 (1997), each entirely incorporated herein by reference).

For the above mentioned procedures, expression libraries usually comprise mammalian cDNA populations, typically human, mouse, simian, or rat, and may represent cDNA produced from RNA of one or more cell type, tissue, or organ and one or more developmental stage. Specific binding for screening cDNA expression libraries is usually provided by including one or more blocking agent (e.g., albumin, nonfat dry milk solids, etc.) prior to and/or concomitant with contacting the labeled hSEZ6 polypeptide (and/or labeled anti-hSEZ6 antibody).

Also included in the present invention are the multitude of screening assays which one skilled in the art can develop to identify compounds which inhibit or induce binding of hSEZ6 to hSEZ6 associating proteins (under suitable binding conditions) based on the disclosures provided herein including, but not limited to, any one of the aforementioned protein-protein interaction assays comprising hSEZ6 polynucleotides, polypeptides, and/or antibodies.

Transgenics and Chimeric Non-Human Mammals

Another embodiment of the present invention provides transgenic non-human mammals carrying a recombinant hSEZ6 gene construct in its somatic and germ cells. The recombinant gene construct may be composed of regulatory DNA sequences that belong to the native hSEZ6 gene or those which are derived from an alternative source. These regulatory sequences are functionally linked to the hSEZ6 coding region, resulting in the constitutive and/or regulatable expression of hSEZ6 in the body of the transgenic non-human mammal. The most important of such regulatory sequences is the promoter. Promoters are defined in this context as any and all DNA elements necessary for the functional expression of a gene. Promoters drive the expression of structural genes and may be modulated by inducers and repressors. Numerous promoters have been described in the literature and are easily within the grasp of the ordinarily skilled artisan. Viral promoters, such as the SV40 early promoter, are consistent with the invention though mammalian promoters are preferred. The promoter is chosen such that the level of expression is sufficient to promote physiological consequences in the transgenic non-human mammal, or ancestor of said mammal. Preferably, the genome of the transgenic mammal contains at least 30 copies of a transgene. More preferably, the genome of the transgenic mammal contains at least 50 copies, and may contain 100-200 or more copies of the transgene. Generally, said nucleic acid is introduced into said mammal at an embryonic stage, preferably the 1-1000 cell or oocyte stage, and, most preferably not later than about the 64-cell stage. Most preferably the transgenic mammal is homozygous for the transgene.

The techniques described in Leder, U.S. Pat. No. 4,736,866 (hereby entirely incorporated by reference) for producing transgenic non-human mammals may be used for the production of a transgenic non-human mammal of the present invention. The various techniques described in U.S. Pat. Nos. 5,454,807, 5,073,490, 5,347,075, 4870,009, and 4,736,866, the entire contents of which are hereby incorporated by reference, may also be used. Such methods are also described in Methods in Molecular Biology, Vol. 18, 1993, Transgenesis Techniques, Principles and Protocols, (Murphy, D., and Carter, D. A.) as well as in U.S. Pat. Nos. 5,174,986, 5,175,383, 5,175,384, and 5,175,385, all of which are herein incorporated by reference.

Also intended to be within the scope of the present invention are chimeric non-human mammals in which fewer than all of the somatic and germ cells contain a DNA construct comprising a nucleic acid encoding a hSEZ6 polypeptide of the present invention. Contemplated chimeric non-human mammals include animals produced when fewer than all of the cells of the morula are transfected in the process of producing the transgenic animal.

Transgenic and chimeric non-human mammals having human cells or tissue engrafted therein are also encompassed by the present invention. Methods for providing chimeric non-human mammals are provided, e.g., in U.S. Ser. Nos. 07/508,225, 07/518,748, 07/529,217, 07/562,746, 07/596,518, 07/574,748, 07/575,962, 07/207,273, 07/241,590 and 07/137,173, which are entirely incorporated herein by reference, for their description of how to engraft human cells or tissue into non-human mammals.

Alternatively, genetic constructs comprising at least one of the hSEZ6 nucleic acid sequences as defined herein may be used to create transgenic “knockouts” of the hSEZ6 gene. Accordingly, the present invention also provides a transgenic animal which has been engineered by homologous recombination to be deficient in the expression of the endogenous SEZ6 gene. Further, the invention provides a method of producing an heterozygous or homozygous transgenic animal deficient in or lacking functional SEZ6 proteins, respectfully, said method comprising:

a) obtaining a DNA construct comprising a disrupted hSEZ6 gene, wherein said disruption is by the insertion of an heterologous marker sequence;

b) introducing said DNA construct into an ES cell of said animal such that the endogenous SEZ6 gene is disrupted by homologous recombination;

c) selecting ES cells comprising said disrupted allele;

d) incorporating the ES cells of step c) into a mouse embryo;

e) transferring said embryo into a pseudopregnant animal of the said species;

f) developing said embryo into a viable offspring;

g) screening offspring to identify heterozygous animal comprising said disrupted SEZ6 gene; and

h) if desired, breeding said heterozygous animal to produce homozygous transgenic animals of said species, wherein the said homozygous animal does not express functional SEZ6 proteins.

Transgenic and chimeric non-human mammals of the present invention may be used for analyzing the consequences of over-expression of at least one hSEZ6 polypeptide in vivo. Such animals are also useful for testing the effectiveness of therapeutic and/or diagnostic agents, either associated or unassociated with delivery vectors or vehicles, which preferentially bind to an hSEZ6 polypeptide of the present invention or act to indirectly modulate hSEZ6 activity hSEZ6 transgenic non-human mammals are useful as an animal models in both basic research and drug development endeavors. Transgenic animals carrying at least one hSEZ6 polypeptide or nucleic acid can be used to test compounds or other treatment modalities which may prevent, suppress, or cure a pathology or disease associated with at least one of the above mentioned hSEZ6 activities. Such transgenic animals can also serve as a model for the testing of diagnostic methods for those same diseases. Furthermore, tissues derived from hSEZ6 transgenic non-human mammals are useful as a source of cells for cell culture in efforts to develop in vitro bioassays to identify compounds that modulate hSEZ6 activity or hSEZ6 dependent signaling. Accordingly, another aspect of the present invention contemplates a method of identifying compounds efficacious in the treatment of at least one previously described disease or pathology associated with SEZ6 activity. A non-limiting example of such a method comprises:

a) generating an hSEZ6 transgenic non-human animal which is, as compared to a wild-type animal, pathologically distinct in some detectable or measurable manner from wild-type version of said non-human mammal;

b) exposing said transgenic animal to a compound, and;

c) determining the progression of the pathology in the treated transgenic animal, wherein an arrest, delay, or reversal in disease progression in transgenic animal treated with said compound as compared to the progression of the pathology in an untreated control animals is indicative that the compound is useful for the treatment of said pathology

Another embodiment of the present invention provides a method of identifying compounds capable of inhibiting hSEZ6 activity in vivo and/or in vitro wherein said method comprises:

a) administering an experimental compound to an hSEZ6 transgenic non-human animal, or tissues derived therefrom, exhibiting one or more physiological or pathological conditions attributable to the overexpression of an hSEZ6 transgene; and

b) observing or assaying said animal and/or animal tissues to detect changes in said physiological or pathological condition or conditions.

Another embodiment of the invention provides a method for identifying compounds capable of overcoming deficiencies in hSEZ6 activity in vivo or in vitro wherein said method comprises:

a) administering an experimental compound to an hSEZ6 transgenic non-human animal, or tissues derived therefrom, exhibiting one or more physiological or pathological conditions attributable to the disruption of the endogenous SEZ6 gene; and

b) observing or assaying said animal and/or animal tissues to detect changes in said physiological or pathological condition or conditions.

Various means for determining a compound's ability to modulate hSEZ6 in the body of the transgenic animal are consistent with the invention. Observing the reversal of a pathological condition in the transgenic animal after administering a compound is one such means. Another more preferred means is to assay for markers of hSEZ6 activity in the blood of a transgenic animal before and after administering an experimental compound to the animal. The level of skill of an artisan in the relevant arts readily provides the practitioner with numerous methods for assaying physiological changes related to therapeutic modulation of hSEZ6 activity.

In all previously described in vitro and in vivo assays, the experimental compound may be administered when applicable, either superficially, orally, parenterally (e.g. by intravenous infusion or injection) or a combination of injection and infusion (iv), intramuscularly (im), or subcutaneously (sc). A preferred route of compound administration to an animal is iv, while oral administration is most preferred.

According to another embodiment the present invention provides a method to induce or inhibit neurite outgrowth, neurite adhesion, neural regeneration, neural degeneration, preventing seizures, reducing frequency and/or severity of seizures, growth-factor mediated chemotaxis, primary or secondary sexual development, or alter behavioral patterns including, but not limited to, sleep or eating disorders wherein said method comprises administering to patient a pharmaceutically acceptable composition comprising a hSEZ6 nucleic acid, polypeptide, antibody, and/or composition as described herein and a pharmaceutically acceptable carrier. The amount of composition utilized in these methods is between about 0.01 and 10 mg/kg body weight/day. A preferred dose is from about 10 to 100 μg/kg of active compound. A typical daily dose for an adult human is from about 0.5 to 100 mg. Preferably, the compositions of this invention should be formulated so that a dosage of between 0.01-10 mg/kg body weight/day of a compound of this invention can be administered. More preferably, the dosage is between 0.1 mg/kg body weight/day. It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular compound of this invention. In practicing this method, compounds of the present invention can be administered in a single daily dose or in multiple doses per day. The treatment regime may require administration over extended periods of time. The amount per administered dose or the total amount administered will be determined by the physician and depend on such factors as the nature and severity of the disease, the age and general health of the patient and the tolerance of the patient to the compound.

The instant invention further provides pharmaceutical formulations comprising a hSEZ6 nucleic acid, polypeptide, and/or anti-hSEZ6 antibody of the present invention. The proteins, preferably in the form of a pharmaceutically acceptable salt, can be formulated for parenteral administration for the therapeutic or prophylactic treatment disorders commonly associated with aberrant hSEZ6 activity. For example, compounds can be admixed with conventional pharmaceutical carriers and excipients. The compositions comprising proteins of the present invention contain from about 0.1 to 95% by weight of the active protein, preferably in a soluble form, and more generally from about 10 to 30%. For intravenous (i.v.) use, said proteins are administered in commonly-used intravenous fluid(s) and administered by infusion. Such fluids, for example, physiological saline, Ringer's solution or 5% dextrose solution can be used. For intramuscular preparations, a sterile formulation, preferably a suitable soluble salt form of the protein, for example the hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate. Pharmaceutically acceptable preservatives such as an alkylparaben, particularly methylparaben, ethylparaben, propylparaben, or butylparaben or chlorobutanol are preferably added to the formulation to allow multi-dose use. Significantly, the claimed proteins are also stable in the presence of a phenolic preservative, such as, m-cresol or phenol. The stability of the proteins in the presence of a phenolic preservative offers advantages in pharmaceutical delivery, including, enhanced preservative effectiveness. The formulation is preferably prepared in the absence of salt to minimize the ionic strength of the formulation.

The pharmaceutical compositions of the present invention maybe administered orally, parenterally, by inhalation spray, nasally, buccally, or via an implanted reservoir. Preferably, the compositions are administered orally, intraperitoneally or intravenously. The pharmaceutical compositions of this invention may contain any conventional non-toxic pharmaceutically-acceptable carriers, adjuvants or vehicles. In some cases, the pH of the formulation maybe adjusted with pharmaceutically acceptable acids, bases or buffers to enhance the stability of the formulated compound or its delivery form. Sterile injectable forms of the compositions of this invention may be aqueous or oleaginous suspension. These suspensions may be formulated according to techniques known in the art using suitable dispersion wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono or di-glycerides. Fatty acids, such as oleic acid and its glyceride derivatives are useful in the preparation of injectables, as are natural pharmaceutically acceptable oils, such as-olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose, corn starch, dicalcium phosphate and microcrystalline cellulose (Avicel). Lubricating agents, such as magnesium stearate and talc, are also typically added. For oral administration in a capsule form, useful diluents include lactose, dried corn starch and TPGS, as well as the other diluents used in tablets. For oral administration in a soft gelatincapsule form (filled with either a suspension or a solution of a compound of this invention), useful diluents include PEG400, TPGS, propylene glycol, Labrasol, Gelucire, Transcutol, PVP and potassium acetate. When aqueous suspensions are administered orally, the active ingredient is combined with emulsifying and suspending agents, such as sodium CMC, methyl cellulose, pectin and gelatin. If desired, certain sweetening and/or flavoring and/or coloring agents may be added.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present invention to produce a single dosage form will vary depending upon the host treated and the particular mode of administration.

The invention further provides for the use of a hSEZ6 agonist, hSEZ6 antagonist, hSEZ6 polypeptide, hSEZ6 nucleic acid, and/or hSEZ6 antibody in the manufacture of a medicament for the treatment or prevention of a disorder in which hSEZ6 activity is detrimental.

The invention further provides for the use of a hSEZ6 agonist, hSEZ6 antagonist, hSEZ6 polypeptide, hSEZ6 nucleic acid, and/or hSEZ6 antibody in the manufacture of a medicament for the treatment or prevention of a disorder in which hSEZ6 activity is detrimental wherein said medicament further comprises another cytokine agonist, antagonist, polypeptide, nucleic acid, and/or antibody.

The invention further provides for the use of a hSEZ6 agonist, hSEZ6 antagonist, hSEZ6 polypeptide, hSEZ6 nucleic acid, and/or hSEZ6 antibody in the manufacture of a medicament for the treatment or prevention of a neurological disorder selected from the group consisting of epilepsy, trigeminal neuralgia, glossopharyngeal neuralgia, Bell's Palsy, myasthenia gravis, muscular dystrophy, muscle injury, progressive muscular atrophy, progressive bulbar inherited muscular atrophy, herniated, ruptured or prolapsed invertebrae disk syndrome, cervical spondylosis, plexus disorders, thoracic outlet destruction syndromes, peripheral neuropathies caused by lead, dapsone, ticks, or porphyria, peripheral myelin disorders, Alzheimer's disease, Gullain-Barre syndrome, Parkinson's disease, Parkinsonian disorders, ALS, multiple sclerosis, central myelin disorders, seizures, stroke, ischemia associated with stroke, neural paropathy, neural degenerative diseases, motor neuron diseases, sciatic crush, neuropathy associated with diabetes, spinal cord trauma, facial nerve crush and other trauma, chemotherapy- or medication-induced neuropathies, and Huntington's disease.

Gene Therapy

Nucleic acids encoding hSEZ6 polypeptides may also be used in gene therapy. In gene therapy applications, genes are introduced into cells in order to achieve in vivo synthesis of a therapeutically effective genetic product, for example, for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where act as inhibitors, despite their low intracellular concentrations caused by their restricted uptake by the cell membrane. Zamecnik et al., Proc. Natl. Acad Sci. USA 83: 4143-4146 [1986]). The oligonucleotides can be modified to enhance their uptake, e.g., by substituting their negatively charged phosphodiester groups by uncharged groups. There are a variety of techniques available for introducing nucleic acids into viable cells. The techniques vary depending upon whether the nucleic acid is transferred into cultured cell in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau, et al., Trends in Biotechnology 11: 205-210 (1991). In some situations it is desirable to provide the nucleic acid source with an agent that targets the target cells, such as an antibody specific for a cell surface membrane protein or the target cell, a ligand for a receptor on the target cells, etc. Where liposomes are employed, proteins which bind to a cell surface membrane protein associated with endocytosis may by used for targeting and/or to facilitate uptake, e.g., capsid proteins or fragments thereof tropic for a particular cell type, antibodies for proteins which undergo internalization in cycling, protein that target intracellular localization and enhance intracellular half-life. The technique of receptor-mediated endocytosis is described, for example by Wu et al., J. Biol. Chem. 262: 4429-4432 (1987). For a review of gene marking and gene therapy protocols see Anderson et al., Science 256: 808-813 (1992).

Having generally described the invention, the same will be more readily understood by reference to the following examples, which are provided by way of illustration and are not intended as limiting.

### EXAMPLE 1

### Expression and Purification of an hSEZ6 Polypeptide in E. coli

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites. These elements are arranged such that a DNA fragment encoding a polypeptide can be inserted in such a way as to produce that polypeptide with the six His residues (i.e., a “6× His tag”) covalently linked to the carboxyl terminus of that polypeptide. However, a polypeptide coding sequence can optionally be inserted such that translation of the six His codons is prevented and, therefore, a polypeptide is produced with no 6× His tag.

The nucleic acid sequence encoding the desired portion of an hSEZ6 polypeptide lacking the hydrophobic leader sequence is amplified from the deposited cDNA clone using PCR oligonucleotide primers (based on the sequences presented, e.g., as presented in at least one of SEQ ID NOS:1 OR 2), which anneal to the amino terminal encoding DNA sequences of the desired portion of an hSEZ6 polypeptide and to sequences in the deposited construct 3′ to the cDNA coding sequence. Additional nucleotides containing restriction sites to facilitate cloning in the pQE60 vector are added to the 5′ and 3′ sequences, respectively.

For cloning an hSEZ6 polypeptide, the 5′ and 3′ primers have nucleotides corresponding or complementary to a portion of the coding sequence of an hSEZ6, e.g., as presented in at least one of SEQ ID NOS:1 or 2, according to known method steps. One of ordinary skill in the art would appreciate, of course, that the point in a polypeptide coding sequence where the 5′ primer begins can be varied to amplify a desired portion of the complete polypeptide shorter or longer than the mature form.

The amplified hSEZ6 nucleic acid fragments and the vector pQE60 are digested with appropriate restriction enzymes and the digested DNAs are then ligated together. Insertion of the hSEZ6 DNA into the restricted pQE60 vector places an hSEZ6 polypeptide coding region including its associated stop codon downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing hSEZ6 polypeptide, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the hSEZ6 is dialyzed against 50 mM Na-acetate buffer pH 6, supplemented with 200 mM NaCl. Alternatively, a polypeptide can be successfully refolded by dialyzing it against 500 mM NaCl, 20% glycerol, 25 mM Tris/HCl pH 7.4, containing protease inhibitors.

If insoluble protein is generated, the protein is made soluble according to known method steps. After renaturation the polypeptide is purified by ion exchange, hydrophobic interaction and size exclusion chromatography. Alternatively, an affinity chromatography step such as an antibody column is used to obtain pure hSEZ6 polypeptide. The purified polypeptide is stored at 4° C. or frozen at −40° C. to −120° C.

### EXAMPLE 2

### Cloning and Expression of an hSEZ6 Polypeptide in a Baculovirus Expression System

As an illustrative example, the plasmid shuttle vector pA2 GP is used to insert the cloned DNA encoding the mature polypeptide into a baculovirus to express an hSEZ6 polypeptide, using a baculovirus leader and standard methods as described in Summers, et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors can be used in place of the vector above, such as pAc373, pVL941 and pAcIM1, as one skilled-in the art would readily appreciate, as long as the construct provides appropriately located signals for transcription, translation, secretion and the like, including a signal peptide and an in-frame AUG as required. Such vectors are described, for instance, in Luckow, et al., Virology 170:31-39.

The cDNA sequence encoding the mature hSEZ6 polypeptide in the deposited or other clone, lacking the AUG initiation codon and the naturally associated nucleotide binding site, is amplified using PCR oligonucleotide primers corresponding to the 5′ and 3′ sequences of the gene. Non-limiting examples include 5′ and 3′ primers having nucleotides corresponding or complementary to a portion of the coding sequence of an hSEZ6 polypeptide, e.g., as presented in at least one of SEQ ID NOS:1 OR 2, according to known method steps.

The amplified fragment is isolated from a 1% agarose gel using a commercially available kit (e.g., “Geneclean,” BIO 101 Inc., La Jolla, Calif.). The fragment then is then digested with the appropriate restriction enzyme and again is purified on a 1% agarose gel. This fragment is designated herein “F1”.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria that contain the plasmid with the human hSEZ6 gene are identified using a PCR method in which one of the primers that is used is designed to amplify the gene and the second primer is from well within the vector so that only those bacterial colonies containing the hSEZ6 gene fragment will show amplification of the DNA. The sequence of the cloned fragment is confirmed by DNA sequencing. This plasmid is designated herein pBacSEZ6.

Five μg of the plasmid pBachSEZ6 is co-transfected with 1.0 μg of a commercially available linearized baculovirus DNA (“BaculoGold™ baculovirus DNA”, Pharmingen, San Diego, Calif.), using the lipofection method described by Felgner, et al., Proc. Natl. Acad. Sci. USA 84:7413-7417 (1987). 1 μg of BaculoGold™ virus DNA and 5 μg of the plasmid pBacSEZ6 are mixed in a sterile well of a microtiter plate containing 50 μl of serum-free Grace's medium (Life Technologies, Inc., Rockville, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, according to known methods. An agarose gel with “Blue Gal” (Life Technologies, Inc., Rockville, Md.) is used to allow easy identification and isolation of gal-expressing clones, which produce blue-stained plaques. (A detailed description of a “plaque assay” of this type can also be found in the user's guide for insect cell culture and baculovirology distributed by Life Technologies, Inc., Rockville, Md., page 9-10). After appropriate incubation, blue stained plaques are picked with a micropipettor tip (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-hSEZ6.

To verify the expression of the hSEZ6 gene, Sf9 cells are grown in Grace's medium supplemented with 10% heat-inactivated FBS. The cells are infected with the recombinant baculovirus V-hSEZ6 at a multiplicity of infection (“MOI”) of about 2. Six hours later the medium is removed and is replaced with SF900 II medium minus methionine and cysteine (available, e.g., from Life Technologies, Inc., Rockville, Md.). If radiolabeled polypeptides are desired, 42 hours later, 5 mCi of 35S-methionine and 5 mCi 35S-cysteine (available from Amersham) are added. The cells are further incubated for 16 hours and then they are harvested by centrifugation. The polypeptides in the supernatant as well as the intracellular polypeptides are analyzed by SDS-PAGE followed by autoradiography (if radiolabeled). Microsequencing of the amino acid sequence of the amino terminus of purified polypeptide can be used to determine the amino terminal sequence of the mature polypeptide and thus the cleavage point and length of the secretory signal peptide.

### EXAMPLE 3

### Cloning and Expression of hSEZ6 in Mammalian Cells

A typical mammalian expression vector contains at least one promoter element, which mediates the initiation of transcription of mRNA, the polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded polypeptide. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### EXAMPLE 3(a)

### Cloning, Expression, and Purification of hSEZ6 Polypeptides in Mammalian Cells

FLAG-HIS (FLIS)-tagged versions of human hSEZ6 polypeptides are expressed in mammalian cells (HEK-293EBNA, COS-7, and/or HEK-293T) to generate recombinant proteins for analysis. Either a pBluescript vector containing an XhoI fragment or a PINCY vector containing a NotI fragment encoding full-length human hSEZ6 polynucloetide is used as a template for PCR amplification of the coding region of the cDNA. Oligonucleotide primers, containing AscI or NheI endonuclease restriction sites for the forward strands and EcoRV, EcoRI, or PmeI restriction sites for the reverse strands, are determined using ordinary skill of the art. The resultant PCR-generated fragment is cleaved with the respective restriction enzymes then gel-purified. The fragment is ligated into a mammalian expression vector that is digested with the appropriate restriction enzymes. Expression vectors which are used include pPR1 (a derivative of pJB02, Berry, J.; Gonzalez-DeWhitt, P.; Ryan, P.; Kovacevic, S.; and Amegadzie, B. Y.; unpublished), pEW1938 (a modified version of pJB02 with a FLIS epitope tag fused to the C-terminus), or pXenoFLIS. The plasmid construct is designed to express a molecule (including the NH2-terminal amino acids which constitute the signal peptide) with the FLIS tag at the COOH-terminus of the protein. Protein expression is controlled by the CMV promoter. For expression of the recombinant hSEZ6 polypeptide, cells are transiently transfected with the expression vector described above. All transfections are performed in spinner culture flasks utilizing cell lines that have been adapted to suspension growth in an animal protein-free medium (APFM). Stock cells are maintained in 6-liter shake flask cultures (130 rpm, 37° C. incubator) at a working volume of approximately 2 liters and a cell density between 0.5 and 3.0×10⁶ cells/mL. For 500 mL transfections, cells from the stock culture are centrifuged, washed, and seeded at 6.0×10⁶ cells/mL into a 1 liter spinner flask containing a total of 450 ML APFM. In a separate container, the DNA are prepared for transfection by adding 250 μg of the appropriate plasmid DNA to 50 mL of APFM, followed by the addition of 500 μL of transfection reagent. A proprietary transfection reagent, X-tremeGENE Ro-1539 (Roche Diagnostics Corp.), is used to introduce DNA into the cells. After a 30 minute incubation at room temperature, the 50 mL DNA per transfection reagent mixture is added to the spinner flask containing the cells. The flask is gassed with a 10% CO₂/air mixture and incubated for 5 days in a non-CO₂ incubator at 37° C. and 150 rpm. After incubation, the cells are removed by centrifugation at 2000×g for 30 minutes, and the conditioned medium is submitted for purification by SDS-PAGE.

High throughput protein isolation is accomplished using affinity chromatography. Under a sterile hood, 1 mL of FLAG affinity resin is added to each flask containing the transfected cells in 500 mL media. The flasks are capped, and the media/resin slurry is shaken on an orbital shaker overnight at 4° C. After shaking, the media is poured into sterile, disposable columns attached to a vacuum manifold located inside a sterile hood. The resin is collected in the columns and washed with sterile, phosphate buffered saline (PBS). The proteins are then eluted with 5 mL of sterile FLAG peptide solution at a concentration of 0.5 mM in PBS. Aliquots of the purified proteins are analyzed by polyacrylamide gel electrophoresis, using electrophoretic molecular weight markers as standards. Densitonetric scanning is used to quantify the proteins in the Coomassie-stained gel. The proteins are further characterized by Western blotting, using the anti-FLAG antibody for detection. The purified protein solutions are stored frozen at −70° C., to be thawed immediately before use in assays.

### EXAMPLE 3(b)

### Cloning and Expression in COS Cells

The expression plasmid, phSEZ6HA, is made by cloning a cDNA encoding hSEZ6 into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eucaryotic cells; (4) a CMV promoter, a polylinker, an SV40 intron; (5) several codons encoding a hemagglutinin fragment (i.e., an “HA” tag to facilitate purification) or HIS tag (see, e.g, Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide described by Wilson, et al., Cell 37:767-778 (1984). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the hSEZ6 is cloned into the polylinker region of the vector so that recombinant polypeptide expression is directed by the CMV promoter. The plasmid construction strategy is as follows. The hSEZ6 cDNA is amplified using primers that contain convenient restriction sites.

The PCR amplified DNA fragment and the vector, pcDNAI/Amp, are digested with suitable restriction enzyme(s) and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the hSEZ6-encoding fragment.

For expression of recombinant hSEZ6, COS cells are transfected with an expression vector, as described above, using DEAE-DEXTRAN, as described, for instance, in Sambrook, et al., Molecular Cloning: a Laboratory Manual, Cold Spring Laboratory Press, Cold Spring Harbor, N.Y. (1989). Cells are incubated under conditions for expression of hSEZ6 by the vector.

Expression of the hSEZ6-HA fusion polypeptide is detected by radio-labeling and immuno-precipitation, using methods described in, for example Harlow, et al., Antibodies: A Laboratory Manual, 2nd Ed., Cold Spring Harbor Laboratory Press, Cold Spring Harbor, N.Y. (1988). To this end, two days after transfection, the cells are labeled by incubation in media containing 35S-cysteine for 8 hours. The cells and the media are collected, and the cells are washed and lysed with detergent-containing RIPA buffer: 150 mM NaCl, 1% NP-40, 0.1% SDS, 0.5% DOC, 50 MM TRIS, pH 7.5, as described by Wilson, et al. cited above. Proteins are precipitated from the cell lysate and from the culture media using an HA-specific monoclonal antibody. The precipitated polypeptides then are analyzed by SDS-PAGE and autoradiography. An expression product of the expected size is seen in the cell lysate, which is not seen in negative controls.

### EXAMPLE 3(c)

### Cloning and Expression in CHO Cells

The vector pC4 is used for the expression of hSEZ6 polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the hSEZ6 in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete hSEZ6 polypeptide is amplified using PCR oligonucleotide primers corresponding to the 5′ and 3′ sequences of the gene as previously described. The amplified fragment is digested with suitable endonucleases and then purified again on a 1% agarose gel. The isolated fragment and the dephosphorylated vector are then ligated with T4 DNA ligase. E. coli HBO101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained which grow at a concentration of 100-200 mM. Expression of the desired gene product is analyzed, for instance, by SDS-PAGE and Western blot or by reverse phase HPLC analysis.

### EXAMPLE 4

### Tissue Distribution of hSEZ6 mRNA Expression

Northern blot analysis is carried out to examine hSEZ6 gene expression in human tissues, using methods described by, among others, Sambrook, et al., cited above. A cDNA probe containing the entire nucleotide sequence of an hSEZ6 polypeptide (SEQ ID NOS:L) is labeled with ³²P using the Rediprime™ DNA labeling system (Amersham Life Science), according to the manufacturer's instructions. After labeling, the probe is purified using a CHROMA SPIN-100™ column (Clontech Laboratories, Inc.), according to the manufacturer's protocol number PT1200-1. The purified and labeled probe is used to examine various human tissues for hSEZ6 mRNA.

Multiple Tissue Northern (MTN) blots containing various human tissues (H) or human immune system tissues (IM) are obtained from Clontech and are examined with the labeled probe using ExpressHyb hybridization solution (Clontech) according to manufacturer's protocol number PT1190-1. Following hybridization and washing, the blots are mounted and exposed to film at −70° C. overnight, and films developed according to standard procedures. The results show hSEZ6 polypeptides to be selectively expressed in neuronal tissues.

### EXAMPLE 5

### Directed Mutagenesis of hSEZ6 Polypeptides to Provide DNA Encoding Specified Substitutions, Insertions or Deletions of SEQ ID NO:1 Using the Polymerase Chain Reaction

The polymerase chain reaction (PCR) can be used for the enzymatic amplification and direct sequencing of small quantities of nucleic acids (see, e.g., Ausubel, supra, section 15) to provide specified substitutions, insertions or deletions in DNA encoding an hSEZ6 polypeptide of the present inventions, e.g., SEQ ID NO:1, 2, 3, or any sequence described herein, as presented herein, to provide an hSEZ6 polypeptide sequence of interest including at least one substitution, insertion or deletion selected from the group consisting of 26I, 27T, 29E, 31H, 33T, 36R, 51S, 52D, 83R, 85E, 87A, 88P, 89Q, 98A, 111T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L,208V, 209E, 213I, 214T, 217G, 235V, 240P, 260A, 261P, 265S, 273Y, 288E, 293Q, 2981, 339L, 380H, 394F, 408Q, 449P, 452S, 477N, 491E, 503R, 509F, 530R, 546A, 548S, 577H, 642S, 667G 690A, 708N, 722N, 749I, 757S, 798V, 806T, 809A, and 835F of SEQ ID NOS:3, or the corresponding amino acids of SEQ ID NOS:4-11. This technology can be used as a quick and efficient method for introducing any desired sequence change into the DNA of interest.

Unit 8.5 of Ausubel, supra, contains two basic protocols for introducing base changes into specific DNA sequences. Basic Protocol 1, as presented in the first section 8.5 of Ausubel, supra (entirely incorporated herein by reference), describes the incorporation of a restriction site and Basic Protocol 2, as presented below and in the second section of Unit 8.5 of Ausubel, supra, details the generation of specific point mutations (all of the following references in this example are to sections of Ausubel et al., eds., Current Protocols in Molecular Biology, Wiley Interscience, New York (1987-1999)). An alternate protocol describes generating point mutations by sequential PCR steps. Although the general procedure is the same in all three protocols, there are differences in the design of the synthetic oligonucleotide primers and in the subsequent cloning and analyses of the amplified fragments.

The PCR procedure described here can rapidly, efficiently, and/or reproducibly introduce any desired change into a DNA fragment. It is similar to the oligonucleotide-directed mutagenesis method described in UNIT 8.1, but does not require the preparation of a uracil-substituted DNA template.

The main disadvantage of PCR-generated mutagenesis is related to the fidelity of the Taq DNA polymerase. The mutation frequency for Taq DNA polymerase was initially estimated to be as high as 1/5000 per cycle (Saiki et al., 1988). This means that the entire amplified fragment must be sequenced to be sure that there are no Taq-derived mutations. To reduce the amount of sequencing required, it is best to introduce the mutation by amplifying as small a fragment as possible. With rapid and reproducible methods of double-stranded DNA sequencing (UNIT 7.4), the entire amplified fragment can usually be sequenced from a single primer. If the fragment is somewhat longer, it is best to subclone the fragment into an M13-derived vector, so that both forward and reverse primers can be used to sequence the amplified fragment.

If there are no convenient restriction sites flanking the fragment of interest, the utility of this method is somewhat reduced. Many researchers prefer the mutagenesis procedure in UNIT 8.1 to avoid excessive sequencing.

A full discussion of critical parameters for PCR amplification can be found in UNIT 15.1.

Literature Cited

Saiki, R. K., Gelfand, D. H., Stoffel, S., Scharf, S. J., Higuchi, R., Horn, G. T., Mullis, K. B., and Erlich, H. A. 1988. Primer-directed enzymatic amplification of DNA with a thermostable DNA polymerase. Science 239:487-491.

Basic Protocol (2): Introduction of Point Mutations by PCR

In this protocol, synthetic oligonucleotides are designed to incorporate a point mutation at one end of an amplified fragment. Following PCR, the amplified fragments are made blunt-ended by treatment with Klenow fragment. These fragments are then ligated and subcloned into a vector to facilitate sequence analysis. This procedure is summarized in FIG. 8.5.2 of Ausubel, supra. Required materials include the DNA sample to be mutagenized, klenow fragment of E. coli DNA polymerase I (UNIT 3.5 of Ausubel, supra), appropriate restriction endonucleases (Table 8.5.1), as well as, the reagents and equipment for synthesis, purification, and phosphorylation of oligonucleotides (UNITS 2.11, 2.12, & 3.10), electrophoresis on nondenaturing agarose and low gelling/melting agarose gels (UNITS 2.5A & 2.6), ligation of DNA fragments (UNIT 3.16), transformation of E. coli (UNIT 1.8), and preparation of plasmid DNA (UNIT 1.6).

Prepare template DNA (see Basic Protocol 1, steps 1 and 2). Synthesize (UNIT 2.11) and purify (UNIT 2.12) the oligonucleotide primers (primers 3 and 4 in FIG. 8.5.2B) accordingly. The oligonucleotide primers must be homologous to the template DNA for more than 15 bases. No four-base “clamp” sequence is added to these primers. The primer sequences are based on a DNA encoding the hSEZ6 polypeptide sequence of interest including at least one substitution, insertion or deletion selected from the group consisting of 26I, 27T, 29E, 31H, 33T, 36R, 51S, 52D, 83R, 85E, 87A, 88P, 89Q, 98A, 111T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L,208V, 209E, 213I, 214T, 217G, 235V, 240P, 260A, 261P, 265S, 273Y, 288E, 293Q, 298I, 339L, 380H, 394F, 408Q, 449P, 452S, 477N, 491E, 503R, 509F, 530R, 546A, 548S, 577H, 642S, 667G 690A, 708N, 722N, 7491, 757S, 798V, 806T, 809A, and 835F of SEQ ID NOS:3, or the corresponding amino acids of SEQ ID NOS:4-11. Phosphorylate the 5′ end of the oligonucleotides (UNIT 3.10). This step is necessary because the 5′ end of the oligonucleotide will be used directly in cloning.

Amplify DNA and Prepare Blunt-End Fragments

Amplify the template DNA (see Basic Protocol 1, steps 5 and 6). After the final extension step, add 5 U Klenow fragment to the reaction mix and incubate 15 min at 30° C. During PCR, the Taq polymerase adds an extra nontemplated nucleotide to the 3′ end of the fragment. The 3′-5′ exonuclease activity of the Klenow fragment is required to make the ends flush and suitable for blunt-end cloning (UNIT 3.5). Analyze and process the reaction mix (see Basic Protocol 1, steps 7 and 8). Digest half the amplified fragments with the restriction endonucleases for the flanking sequences (UNIT 3.1). Purify digested fragments on a low gelling/melting agarose gel (UNIT 2.6).

Subclone the two amplified fragments into an appropriately digested vector by blunt-end ligation (UNIT 3.16). Transform recombinant plasmid into E. coli (UNIT 1.8). Prepare DNA by plasmid miniprep (UNIT 1.6). Analyze the amplified fragment portion of the plasmid DNA by DNA sequencing to confirm the point mutation (UNIT 7.4). This is critical because the Taq DNA polymerase can introduce additional mutations into the fragment (see Critical Parameters).

Alternate Protocol: Introduction of a Point Mutation by Sequential PCR Steps

In this procedure, the two fragments encompassing the mutation are annealed with each other and extended by mutually primed synthesis; this fragment is then amplified by a second PCR step, thereby avoiding the blunt-end ligation required in Basic Protocol 2. This strategy is outlined in FIG. 8.5.3. For materials, see Basic Protocols 1 and 2 of Ausubel, supra.

Prepare template DNA (see Basic Protocol 1, steps 1 and 2). Synthesize (UNIT 2.11) and purify (UNIT 2.12) the oligonucleotide primers (primers 5 and 6 in FIG. 8.5.3B) to generate an hSEZ6 polypeptide sequence of interest including at least one substitution, insertion or deletion selected from the group consisting of 26I, 27T, 29E, 31H, 33T, 36R, 51S, 52D, 83R, 85E, 87A, 88P, 89Q, 98A, 11T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L, 208V, 209E, 213I, 214T, 217G, 235V, 240P, 260A, 261P, 265S, 273Y, 288E, 293Q, 2981, 339L, 380H, 394F, 408Q, 449P, 452S, 477N, 491E, 503R, 509F, 530R, 546A, 548S, 577H, 642S, 667G 690A, 708N, 722N, 749I, 757S, 798V, 806T, 809A, and 835F of SEQ ID NOS:3, or the corresponding amino acids of SEQ ID NOS:4-11. The oligonucleotides must be homologous to the template for 15 to 20 bases and must overlap with one another by at least 10 bases. The 5′ end does not have a “clamp” seguence.

Amplify the template DNA and generate blunt-end fragments (see Basic Protocol 2, steps 4 and 5). Purify the fragments by nondenaturing agarose gel electrophoresis (UNIT 2.5A). Resuspend in TE buffer at 1 ng/ul.

Carry out second PCR amplification. Combine the following in a 500-ul microcentrifuge tube:

10 μl (10 ng) each amplified fragment

1 μl (500 ng) each flanking sequence primer (each 1 μM final)

10 μl 10× amplification buffer

10 μl 2 mM 4dNTP mix

H₂O to 99.5 μl

0.5 μl Taq DNA polymerase (5 U/μl).

Overlay with 100 μl mineral oil. Carry out PCR for 20 to 25 cycles, using the conditions for introduction of restriction endonuclease sites by PCR (see Basic Protocol 1, step 6). Analyze and process the reaction mix (see Basic Protocol 1, Ausubel, supra, steps 7 and 8).

Digest the DNA fragment with the appropriate restriction endonuclease for the flanking sites (UNIT 3.1). Purify the digested fragment on a low gelling/melting agarose gel (UNIT 2.6). Subclone into an appropriately digested vector. Transform recombinant plasmid into E. coli (UNIT 1.8). Prepare DNA by plasmid miniprep (UNIT 1.6). Analyze the amplified fragment portion of the plasmid DNA by DNA sequencing (UNIT 7.4) to confirm the point mutation. This is critical because the Taq DNA polymerase can introduce additional mutations into the fragment (see Critical Parameters).

### EXAMPLE 6

### Protein Phosphorylation on Tyrosine Residues

Protein-induced cell responses are determined by monitoring tyrosine phosphorylation upon stimulation of cells by addition of hSEZ6 proteins. This is accomplished in two steps: cell manipulation and immunodetection.

Protein phosphorylation was measured using the following cell lines:

GH4C1 (ATCC CCL-82.2)

LNCAP (ATCC CRL-1740)

SK-N-MC (ATCC HTB-10)

U373MG, MCF-7 (ATCC HTB-22)

HM3

ECV304 (endothelial cell line)

GLUTag (SV40 Tag transformed enteroendocrine cell line)

BTC6 (insulinoma cell line)

TF.1 (ATCC CRL-2003)

balb/c 3T3 (ATCC CCL-163)

HDF (dermal fibroblasts) (Clonetics #CC251T150)

M07E (leukemia cell line)

On day 1, the cells are plated into poly-D-lysine-coated, 96 well plates containing cell propagation medium [DMEM:F12 (3:1), 20 mM Hepes at pH 7.5, 5% FBS, and 50 μg/mL Gentamicin]. The cells are seeded at a concentration of 20,000 cells per well in 100 μL medium. On day 2, the propagation medium in each well is replaced with 100 μL starvation medium containing DMEM:F12 (3:1), 20 mM Hepes at pH 7.5, 0.5% FBS, and 50 μg/mL Gentamicin. The cells are incubated overnight.

On day three, pervanadate solution is made 10 minutes before cell lysis; pervanadate is prepared by mixing 100 μL of sodium orthovanadate (100 mM) and 3.4 μL of H₂O₂ (producing 100× stock pervanadate solution). The lysis buffer is then prepared: 50 mM Hepes at pH 7.5, 150 mM NaCl, 10% glycerol, 1% TRITON X-100, 1 mM EDTA, 1 mM pervanadate, and BM protease inhibitors. The cells are stimulated by adding 10 μL of an hSEZ6 protein solution to the cells, and incubating for 10 minutes. Next, the medium is aspirated, and 75 μL lysis buffer are added to each well. The cells are lysed at 4° C. for 15 minutes, then 25 μL of 4× loading buffer are added to the cell lysates. The resultant solution is mixed then heated to 95° C.

Detection of tyrosine phosphorylation is accomplished by Western immunoblotting. Twenty microliters of each cell sample are loaded onto SDS-PAGE 8-16% AA ready gels from Bio-Rad, and-the gels are run. The proteins are electrotransferred in transfer buffer (25 mM Tris base at pH 8.3, 0.2 M glycine, 20% methanol) from the gel to a nitrocellulose membrane using 250 mA per gel over a one hour period. The membrane is incubated for one hour at ambient conditions in blocking buffer consisting of TBST (20 mM TrisHCl at pH 7.5, 150 mM NaCl, 0.1% TWEEN-20) with 1% BSA.

Next, the antibodies are added to the membrane. The membrane is incubated overnight at 4° C. with gentle rocking in primary antibody solution consisting of the antibody, TBST, and 1% BSA. The next day, the membrane is washed three times, five minutes per wash, with TBST. The membrane is then incubated in the secondary antibody solution consisting of the antibody, TBST, and 1% BSA for 1 hour at ambient conditions with gentle rocking. After the incubation, the membrane is washed four times with TBST, ten minutes per wash.

Detection is accomplished by incubating the membrane with 10 to 30 mL of SuperSignal Solution for 1 minute at ambient conditions. After 1 minute, excess developing solution is removed, and the membrane is wrapped in plastic wrap. The membrane is exposed to X-ray film for 20 second, 1 minute, and 2 minute exposures (or longer if needed). The number and intensity of immunostained protein bands are compared to bands for the negative control-stimulated cells (basal level of phosphorylation) by visual comparison. GLUTag cells stimulated with hSEZ6 polypeptide induced tyrosine phosphorylation in those cells.

### EXAMPLE 7

### Cell Stimulation with Detection Utilizing Reporters

Protein-induced cell responses are measured using reporters. The following cell line/reporter combinations are used:

| cell | reporter element |
|---|---|
| GH4C1 (ATCC CCL-82.2) | pan-STAT |
| LNCAP (ATCC CRL-1740) | pan-STAT |
| L6 (ATCC CRL-1458) | AP-1 |
| SK-N-MC (ATCC HTB-10) | pan-STAT |
| HM3 | pan-STAT |
| GLUTag (SV40 Tag transformed | CRE4 |
| enteroendocrine cell line) | |

For each reporter used, positive controls are designed in the form of agonist cocktails. These cocktails included approximate maximal stimulatory doses of several ligands known to stimulate the regulated signal pathway. The following agonist cocktails are used as positive controls:

| element | pathway | agonist cocktail |
|---|---|---|
| CRE4 | cAMP | Forskolin, isoproteranol, PGE2 |
| pan-STAT | Jak-STAT | IFNα, GCSF, leptin, EPO |
| AP-1 | MAP-kinase | thrombin, PDGF, TNFα, EGF |

Cell lines and reporters with no exogenous stimulus added are used as negative controls.

At time zero, the cells are transiently transfected with the reporter plasmids in tissue culture flasks using a standard optimized protocol for all cell lines (see Example 1). After 24 hours, the cells are trypsinized and seeded into 96-well poly-D-lysine coated assay plates at a rate of 20,000 cells per well in growth medium. After four to five hours, the medium is replaced with serum-free growth medium. At that time, stimulants for those reporters which required a 24-hour stimulation period are added. After 48 hours, stimulants for the reporters which required a 5-hour stimulation period are added. Five hours later, all conditions are lysed using a lysis/luciferin cocktail, and the fluorescence of the samples is determined using a Micro Beta reader.

Each assay plate is plated to contain 4 positive control wells, 16 negative control wells, and 64 test sample wells (2 replicates of 32 test samples). The threshold value for a positive “hit” is a fluorescence signal equal to the mean plus two standard deviations of the negative control wells. Any test sample that, in both replicates, generates a signal above that threshold is defined as a “hit.”

### EXAMPLE 8

### Cell Proliferation and Cytotoxicity Determination Utilizing Fluorescence Detection

This assay is designed to monitor gross changes in the number of cells remaining in culture after exposure to hSEZ6 proteins for a period of three days. The following cells are used in this assay:

Saos (osteosarcoma cell line)

LNCAP (ATCC CRL-1740)

SK-N-MC (ATCC HTB-10)

U373MG, MCF-7 (ATCC HTB-22)

GLUTag (SV40 Tag transformed enteroendocrine cell line)

HUVEC (Clonetics #CC2517T150)

TF.1 (ATCC CRL-2003)

HDF (dermal fibroblasts) (Clonetics #CC2511T150)

T1165 (B cell line)

Prior to assay, cells are incubated in an appropriate assay medium to produce a sub-optimal growth rate, e.g., a 1:10 or 1:20 dilution of normal culture medium. Cells are grown in T-150 flasks, then harvested by trypsin digestion and replated at 40 to 50% confluence into poly-D-lysine-treated 96-well plates. Cells are only plated into the inner 32 wells to prevent edge artifacts due to medium evaporation; the outer wells are filled with buffer alone. Following incubation overnight to stabilize cell recovery, hSEZ6 proteins are added to the appropriate wells. Each protein is assayed in triplicate at two different concentrations, 1× and 0.1× dilution in assay medium. Two controls are also included on each assay plate: assay medium and normal growth medium. After approximately 72 hours of exposure, the plates are processed to determine the number of viable cells. Plates are spun to increase the attachment of cells to the plate. The medium is then discarded, and 50 μL of detection buffer is added to each well. The detection buffer consisted on MEM medium containing no phenol red (Gibco) with calcein AM (Molecular Probes) and PLURONIC™ F-127 (Molecular Probes), each at a 1:2000 dilution. After incubating the plates in the dark at room temperature for thirty minutes, the fluorescence intensity of each well is measured using a Cytofluor 4000-plate reader (PerSeptive Biosystems). For a given cell type, the larger the fluorescence intensity, the greater the number of cells in the well. To determine the effects on cell growth from each plate, the intensity of each well containing cells stimulated with an hSEZ6 protein is subtracted from the intensity of the wells containing assay medium only (controls). Thus, a positive number indicated stimulation of cell growth; a negative number indicated a reduction in growth. Additionally, confidence limits at 95 and 90% are calculated from the mean results. Results lying outside the 95% confidence limit are scored as “definite hits.” Results lying between the 95 and 90% confidence limits are scored as “maybes.” The distinction between definite hits and maybes varied due to intraplate variability; thus, subjective scoring is used as a final determination for “hits.” Cell assays performed using hSEZ6 polypeptide showed increased proliferation of SK-N-MC neuroblastoma cells and MCF7 breast cancer cells.

### EXAMPLE 9

### Determination of Protein Binding in Human Tissue

Binding of hSEZ6 proteins to human tissues is determined by protein staining with fluorescent dye. The following human tissues are used in this assay:

| | |
|---|---|
| liver | hepatocytes |
| pancreas | Islet cells |
| | Acinar cells |
| gut | gastric epithelial cells |
| | crypt epithelial cells |
| | Brunner's Glands |
| | muscularis (small intestine and |
| | colon) |
| ampulla (oviduct) | epithelial cells |
| | muscularis |
| bone marrow | heme stem cells |
| prostate | epithelium |
| uterus | myometrium |
| skin | epidermis |
| breast | ductile epithelial cells |

All tissues are fixed with 3% paraformaldehyde and embedded in paraffin. Tissues are prepared for analysis by removing the paraffin with xylene then gradually rehydrating the tissue with graded solutions of ethanol and water. Antigen retrieval is performed to unmask antigenic sites so that antibodies can recognize the antigen. This is accomplished by soaking the tissue in citrate buffer (Dako, Carpinteria, Calif.) for twenty minutes at 80 to 90° C. followed 10 minutes at ambient temperature. The tissue is then washed in tris-buffered saline (TBS) containing 0.05% TWEEN™ 20 and 0.01% thimerosol. To minimize non-specific background staining, the tissue is soaked in non-serum protein block (Dako) for 45 minutes, after which the protein block is removed by blowing air over the tissue.

The tissue is exposed for 2 hours to the FLAG-HIS tagged hSEZ6 protein at 10 μg/mL. Following exposure, the tissue is washed twice with tris-buffered saline (TBS) containing 0.05% TWEEN™ 20 and 0.01% thimerosol. The tissue sample is then incubated for one hour with mouse anti-FLAG antibody at 10 μg/mL. Subsequently, the tissue is washed twice with tris-buffered saline (TBS) containing 0.05% TWEEN™ 20 and 0.01% thimerosol. Next, the tissue is exposed to rabbit anti-mouse Ig with Alexa 568, a fluorescent dye, at 10 μg/mL for one hour, followed again by two washes with tris-buffered saline (TBS) containing 0.05% TWEEN™ 20 and 0.01% thimerosol. Finally, the tissue is coverslipped with fluorescence mounting media, and the fluorescence is measured. A positive fluorescence reading indicates that the protein binds with antigens on the tissue, suggesting that the protein is expressed in that tissue.

Fluorescently-tagged hSEZ6 stained in the small intestine tissues (including Brunner's Glands) and the islet cells of the pancreas, indicating expression of hSEZ6 in those tissues.

It will be clear that the present invention can be practiced otherwise than as particularly described in the foregoing description and examples. Numerous modifications and variations of the present invention are possible in light of the above teachings and, therefore, are within the scope of the appended claims.

### EXAMPLE 10

### Treatment or Prevention of Neurological Diseases or Neuropathologies with hSEZ6 Polypeptides

This protocol is a controlled trial in patients with a seizure disorder which displays many hallmarks of epilepsy and is treated with hSEZ6 polypeptide or fragment or variant thereof as described herein.

For epilepsy, the attending physician administers hSEZ6 polypeptide or fragment or variant thereof subcutaneously at a dose of 0.5 mg/day, to ensure a slower release into the bloodstream. The treatment is continued until the patient is relieved of the symptoms of the disorder.

Another protocol is a controlled trial in patients with Parkinson's Disease and is treated with hSEZ6 polypeptide or fragment or variant thereof as described herein.

For Parkinson's Disease, the attending physician administers hSEZ6 polypeptide or fragment or variant thereof subcutaneously at a dose of 0.5 mg/day, to ensure a slower release into the bloodstream. The treatment is continued until the patient is relieved of the symptoms of the disorder.

Yet another protocol is a controlled trial in patients with Alzhemier's Disease and is treated with hSEZ6 polypeptide or fragment or variant thereof as described herein.

For Alzheimer's Disease, the attending physician administers hSEZ6 polypeptide or fragment or variant thereof subcutaneously at a dose of 0.5 mg/day, to ensure a slower release into the bloodstream. The treatment is continued until the patient is relieved of the symptoms of the disorder.

For all of the above mentioned treatment protocols, the particular dose of hSEZ6 polypeptide or fragment or variant thereof administered and the route of administration is adjusted by the attending physician evaluating the particular circumstances surrounding the case, including the compound administered, the particular condition being treated, the patient characteristics and similar considerations.

What is claimed is:

1. An isolated nucleic acid comprising at least one hSEZ6 polynucleotide encoding a protein sequence selected from the group of sequences of SEQ ID NOS:3-11, and any fragment thereof.

2. The isolated nucleic acid of claim 1, further comprising at least one mutation corresponding to at least one substitution, insertion, or deletion selected from the group consisting of 26I, 27T, 29E, 31H, 33T, 36R, 51S, 52D, 83R, 85E, 87A, 88P, 89Q, 98A, 111T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L,208V, 209E, 213I, 214T, 217G, 235V, 240P, 260A, 261P, 265S, 273Y, 288E, 293Q, 298I, 339L, 380H, 394F, 408Q, 449P, 452S, 477N, 491E, 503R, 509F, 530R, 546A, 548S, 577H, 642S, 667G 690A, 708N, 722N, 749I, 757S, 798V, 806T, 809A, and 835F of SEQ ID NO:3 or the corresponding amino acid of SEQ ID NOS: 4-11.

3. An isolated nucleic acid complementary to the polynucleotide of claim 1 or 2.

4. A composition comprising at least one isolated nucleic acid according to any one of claims 1-3 and a carrier or diluent.

5. A recombinant vector comprising at least one nucleic acid according to any of claims 1-3.

6. A host cell comprising at least one recombinant vector according to claim 5.

7. A method for producing at least one hSEZ6 polypeptide comprising culturing a host cell according to claim 6 under conditions that the at least one hSEZ6 polypeptide is expressed in detectable or recoverable amounts.

8. A transgenic or chimeric non-human animal comprising at least one isolated nucleic acid according to any of claims 1-3.

9. An isolated polypeptide comprising a polypeptide selected from the group of polypeptide sequences as shown in SEQ ID NOS:3-11, and any fragment thereof.

10. The isolated polypeptide according to claim 9, further comprising at least one mutation corresponding to at least one substitution, insertion, or deletion selected from the group consisting of 261, 27T, 29E, 31H, 33T, 36R, 51S, 52D, 83R, 85E, 87A, 88P, 89Q, 98A, 11T, 115N, 126V, 129A, 134H, 136R, 138K, 141N, 142L, 145K, 146P, 148E, 150S, 153S, 154S, 167L, 169E, 171R, 172P, 179Q, 192D, 197P, 200M 202K, 203T, 204T, 206L,208V, 209E, 213I, 214T, 217G, 235V, 240P, 260A, 261P, 265S, 273Y, 288E, 293Q, 298I, 339L, 380H, 394F, 408Q, 449P, 452S, 477N, 491E, 503R, 509F, 530R, 546A, 548S, 577H, 642S, 667G 690A, 708N, 722N, 7491, 757S, 798V, 806T, 809A, and 835F of SEQ ID NO:3 or the corresponding amino acid of SEQ ID NOS: 4-11.

11. An isolated polypeptide comprising at least one polypeptide comprising at least 90-100% of the contiguous amino acids of at least one extracellular, intracellular, transmembrane or active domain of at least one of SEQ ID NOS:3-11.

12. A pharmaceutical composition comprising at least one isolated polypeptide according to any of claims 9-11 and a carrier or diluent.

13. An isolated nucleic acid probe, fragment, or primer, comprising an hSEZ6 polynucleotide comprising a sequence corresponding or complementary to at least 50 nucleotides of SEQ ID NOS:1 or 2.

14. An isolated nucleic acid comprising a nucleic acid that hybridizes under high stringency conditions to a nucleic acid according to claim 13.

15. An antibody or at least one fragment thereof that binds an epitope specific to at least one hSEZ6 polypeptide according to any of claims 8-11.

16. A host cell, expressing at least one antibody or at least one fragment thereof according to claim 15.

17. A method for producing at least one antibody, comprising culturing a host cell according to claim 16.

18. A method for identifying compounds that bind at least one hSEZ6 polypeptide, comprising
(a) admixing at least one isolated hSEZ6 polypeptide according to any of claims 8-11 with at least one test compound or composition; and
(b) detecting at least one binding interaction between said at least one hSEZ6 polypeptide and the test compound or composition.

19. A compound or composition detected by method according to claim 18.

20. A method for enhancing neuronal growth, neurite outgrowth, neuronal regeneration, or neuronal survival in a patient or in an ex vivo nerve cell comprising the step of administering to said patient or said nerve cell an effective amount of a composition according to claim 12.

21. A method for treating a patient suffering from a neurological disorder comprising the step of administering to said patient an effective amount of a composition according to claim 12.

22. The method for treating a patient suffering from a neurological disorder according to claim 21 wherein said neurological disorder is epilepsy or seizure related.

23. The method for treating a patient suffering from a neurological disorder according to claim 21 wherein said neurological disorder is Alzheimer's disease.

24. The method for treating a patient suffering from a neurological disorder according to claim 21 wherein said neurological disorder is Parkinson's disease.

25. The method for treating a patient suffering from a neurological disorder according to claim 21 wherein said neurological disorder is associated with stroke.

26. The use of a hSEZ6 agonist, hSEZ6 antagonist, hSEZ6 polypeptide, hSEZ6 nucleic acid, and/or hSEZ6 antibody for the manufacture of a medicament for the treatment or prevention of seizures in a mammal.

27. The use of claim 26, wherein the hSEZ6 agonist, hSEZ6 antagonist, hSEZ6 polypeptide, hSEZ6 nucleic acid, and/or hSEZ6 antibody is administered to the mammal together with a cytokine agonist, antagonist, polypeptide, nucleic acid, and/or antibody.

28. A pharmaceutical formulation comprising as an active ingredient a hSEZ6 polypeptide as claimed in any one claims 9 to 11, associated with one or more pharmaceutically acceptable carriers, excipients, or diluents therefore.


SEQUENCE LISTING (11 sequences)

SEQ ID NO: 1 - DNA, 4198 bp, Homo sapiens
cccccggccgtcgccaggcgctggccgtggtgctgattctgtcaggcgctggcggcggcagcggcggtgacggctgcggccccgctccctctacccggccggacccggctctgcccccgcgcccaagccccaccaagccccccgccctcccgccgcggtcccagcccagggcgcggccgcaaccagcaccatgcgcccggtagccctgctgctcctgccctcgctgctggcgctcctggctcacggactctctttagaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagctgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcagggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcctgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtgaaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaagtagctacacccagcaggggccaccatccacttctcgtgtgcccctggctatgtgctgaagggccaggccagcatcaagtgtgtgcctgggcacccctcgcattggagtgaccccccacccatctgtagggctgcctctctggatgggttctacaacagtcgcagcctggatgttgccaaggcacctgctgcctccagcaccctggatgctgcccacattgcagctgccatcttcttgccactggtggcgatggtgttgttggtaggaggtgtatacttctacttctccaggctccagggaaaaagctccctgcagctgccccgcccccgcccccgcccctacaaccgcattaccatagagtcagcgtttgacaatccaacttacgagactggagagacgagagaatatgaagtctccatctaggtgggggcagtctagggaagtcaactcagacttgcaccacagtccagcagcaaggctccttgcttcctgctgtccctccacctcctgtatataccacctaggaggagatgccaccaagccctcaagaagttgtgcccttccccgcctgcgatgcccaccatggcctattttcttggtgtcattgcccacttggggcccttcattgggcccatgtcagggggcatctacctgtgggaagaacatagctggagcacaagcatcaacagccagcatcctgagcctcctcatgccctggaccagcctggaacacactagcagagcaggagtacctttctccacatgaccaccatcccgccctggcatggcaacctgcagcaggattaacttgaccatggtgggaactgcaccagggtactcctcacagcgccatcaccaatggccaaaactcctctcaacggtgacctctgggtagtcctggcatgccaacatcagcctcttgggaggtctctagttctctaaagttctggacagttctgcctcctgccctgtcccagtggaggcagtaattctaggagatcctaaggggttcagggggaccctacccccacctcaggttgggcttccctgggcactcatgctccacaccaaagcaggacacgccattttccactgaccaccctataccctgaggaaagggagactttcctccgatgtttatttagctgttgcaaacatcttcaccctaatagtccctcctccaattccagccacttgtcaggctctcctcttgaccactgtgttatgggataaggggagggggtgggcatattctggagaggagcagaggtccaaggacccaggaatttggcatggaacaggtggtaggagagccccagggagacgcccaggagctggctgaaagccactttgtacatgtaatgtattatatggggtctgggctccagccagagaacaatcttttatttctgttgtttccttattaaaatggtgtttttggaaaaaaaa

SEQ ID NO: 2 - DNA, 2559 bp, Homo sapiens
atgcgcccggtagccctgctgctcctgccctcgctgctggcgctcctggctcacggactctctttagaggccccaaccgtggggaaaggacaagccccaggcatcgaggagacagatggcgagctgacagcagcccccacacctgagcagccagaacgaggcgtccactttgtcacaacagcccccaccttgaagctgctcaaccaccacccgctgcttgaggaattcctacaagaggggctggaaaagggagatgaggagctgaggccagcactgcccttccagcctgacccacctgcacccttcaccccaagtccccttccccgcctggccaaccaggacagccgccctgtctttaccagccccactccagccatggctgcggtacccactcagccccagtccaaggagggaccctggagtccggagtcagagtcccctatgcttcgaatcacagctcccctacctccagggcccagcatggcagtgcccaccctaggcccaggggagatagccagcactacaccccccagcagagcctggacaccaacccaagagggtcctggagacatgggaaggccgtgggttgcagaggttgtgtcccagggcgcagggatcgggatccaggggaccatcacctcctccacagcttcaggagatgatgaggagaccaccactaccaccaccatcatcaccaccaccatcaccacagtccagacaccaggcccttgtagctggaatttctcaggcccagagggctctctggactcccctacagacctcagctcccccactgatgttggcctggactgcttcttctacatctctgtctaccctggctatggcgtggaaatcaaggtccagaatatcagcctccgggaaggggagacagtgactgtggaaggcctgggggggcctgacccactgcccctggccaaccagtctttcctgctgcggggccaagtcatccgcagccccacccaccaagcggccctgaggttccagagcctcccgccaccggctggccctggcaccttccatttccattaccaagcctatctcctgagctgccactttccccgtcgtccagcttatggagatgtgactgtcaccagcctccacccagggggtagtgcccgcttccattgtgccactggctaccagctgaagggcgccaggcatctcacctgtctcaatgccacccagcccttctgggattcaaaggagcccgtctgcatcgctgcttgcggcggagtgatccgcaatgccaccaccggccgcatcgtctctccaggcttcccgggcaactacagcaacaacctcacctgtcactggctgcttgaggctcctgagggccagcggctacacctgcactttgagaaggtttccctggcagaggatgatgacaggctcatcattcgcaatggggacaacgtggaggccccaccagtgtatgattcctatgaggtgaaatacctgcccattgagggcctgctcagctctggcaaacacttctttgttgagctcagtactgacagcagcggggcagctgcaggcatggccctgcgctatgaggccttccagcagggccattgctatgagccctttgtcaaatacggtaacttcagcagcagcacacccacctaccctgtgggtaccactgtggagttcagctgcgaccctggctacaccctggagcagggctccatcatcatcgagtgtgttgacccccacgacccccagtggaatgagacagagccagcctgccgagccgtgtgcagcggggagatcacagactcggctggcgtggtactctctcccaactggccagagccctacggtcgtgggcaggattgtatctggggtgtgcatgtggaagaggacaagcgcatcatgctggacatccgagtgctgcgcataggccctggtgatgtgcttaccttctatgatggggatgacctgacggcccgggttctgggccagtactcagggccccgtagccacttcaagctctttacctccatggctgatgtcaccattcagttccagtcggaccccgggacctcagtgctgggctaccagcagggcttcgtcatccacttctttgaggtgccccgcaatgacacatgtccggagctgcctgagatccccaatggctggaagagcccatcgcagcctgagctagtgcacggcaccgtggtcacttaccagtgctaccctggctaccaggtagtgggatccagtgtcctcatgtgccagtgggacctaacttggagtgaggacctgccctcatgccagagggtgacttcctgccacgatcctggagatgtggagcacagccgacgcctcatatccagccccaagtttcccgtgggggccaccgtgcaatatatctgtgaccagggttttgtgctgatgggcagctccatcctcacctgccatgatcgccaggctggcagccccaagtggagtgaccgggcccctaaatgtctcctggaacagctcaagccatgccatggtctcagtgcccctgagaatggtgcccgaagtcctgagaag

SEQ ID NO: 3 - protein, 853 aa, Homo sapiens
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVKYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEK

SEQ ID NO: 4 - protein, 829 aa, Homo sapiens
PTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVKYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEK

SEQ ID NO: 5 - protein, 24 aa, Homo sapiens
MRPVALLLLPSLLALLAHGLSLEA

SEQ ID NO: 6 - protein, 56 aa, Homo sapiens
CHFPRRPAYGDVTVTSLHPGGSARFHCATGYQLKGARHLTCLNATQPFWDSKEPVC

SEQ ID NO: 7 - protein, 58 aa, Homo sapiens
CYEPFVKYGNFSSSTPTYPVGTTVEFSCDPGYTLEQGSIIIECVDPHDPQWNETEPAC

SEQ ID NO: 8 - protein, 56 aa, Homo sapiens
CPELPEIPNGWKSPSQPELVHGTVVTYQCYPGYQVVGSSVLMCQWDLTWSEDLPSC

SEQ ID NO: 9 - protein, 60 aa, Homo sapiens
CHDPGDVEHSRRLISSPKFPVGATVQYICDQGFVLMGSSILTCHDRQAGSPKWSDRAPKC

SEQ ID NO: 10 - protein, 109 aa, Homo sapiens
CGGVIRNATTGRIVSPGFPGNYSNNLTCHWLLEAPEGQRLHLHFEKVSLAEDDDRLIIRNGDNVEAPPVYDSYEVKYLPIEGLLSSGKHFFVELSTDSSGAAAGMALRY

SEQ ID NO: 11 - protein, 109 aa, Homo sapiens
CSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHF